SEQUENCE LISTING (22 sequences)

SEQ ID NO: 1 - protein, 317 aa, Homo sapiens [sequence omitted]

SEQ ID NO: 2 - protein, 299 aa, Homo sapiens
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 3 - RNA, 21 nt, Artificial Sequence
  Description of Artificial Sequence Synthetic oligonucleotide
gucaucacacugaauaccaau

SEQ ID NO: 4 - RNA, 23 nt, Artificial Sequence
  Description of Artificial Sequence Synthetic oligonucleotide
auugguauucagugugaugacac

SEQ ID NO: 5 - DNA, 21 bp, Artificial Sequence
  Description of Artificial Sequence Synthetic oligonucleotide
agucaaucgguauguccactt

SEQ ID NO: 6 - DNA, 21 bp, Artificial Sequence
  Description of Artificial Sequence Synthetic oligonucleotide
guggacauaccgauugacutt

SEQ ID NO: 7 - DNA, 21 bp, Artificial Sequence
  Description of Artificial Sequence Synthetic oligonucleotide
agucagucaguguauccactt

SEQ ID NO: 8 - DNA, 21 bp, Artificial Sequence
  Description of Artificial Sequence Synthetic oligonucleotide
guggauacacugacugacutt

SEQ ID NO: 9 - DNA, 19 bp, Artificial Sequence
  Description of Artificial Sequence Synthetic oligonucleotide
gcagcaactgatatttgta

SEQ ID NO: 10 - DNA, 19 bp, Artificial Sequence
  Description of Artificial Sequence Synthetic oligonucleotide
agctggtcattacctatca

SEQ ID NO: 11 - DNA, 24 bp, Artificial Sequence
  Description of Artificial Sequence Synthetic primer
ctcctgttgcagtagagtgcagct

SEQ ID NO: 12 - DNA, 30 bp, Artificial Sequence
  Description of Artificial Sequence Synthetic primer
ctctagagcgactggagcacgaggacacta

SEQ ID NO: 13 - DNA, 38 bp, Artificial Sequence
  Description of Artificial Sequence Synthetic primer
acgcgtcgacgtgggagcatggaggttggcagttgttc

SEQ ID NO: 14 - RNA, 21 nt, Artificial Sequence
  Description of Artificial Sequence Synthetic oligonucleotide
gucaucacacugaauaccaau

SEQ ID NO: 15 - RNA, 23 nt, Artificial Sequence
  Description of Artificial Sequence Synthetic oligonucleotide
auugguauucagugugaugacac

SEQ ID NO: 16 - RNA, 21 nt, Artificial Sequence
  Description of Artificial Sequence Synthetic oligonucleotide
ggaaucuuauauuugauccaa

SEQ ID NO: 17 - RNA, 23 nt, Artificial Sequence
  Description of Artificial Sequence Synthetic oligonucleotide
uuggaucaaauauaagauucccu

SEQ ID NO: 18 - DNA, 21 bp, Artificial Sequence
  Description of Artificial Sequence Synthetic oligonucleotide
gccuggaguuuauucggaatt

SEQ ID NO: 19 - DNA, 21 bp, Artificial Sequence
  Description of Artificial Sequence Synthetic oligonucleotide
uuccgaauaaacuccaggctt

SEQ ID NO: 20 - DNA, 21 bp, Artificial Sequence
  Description of Artificial Sequence Synthetic oligonucleotide
ggaucaucucaagucuuactt

SEQ ID NO: 21 - DNA, 21 bp, Artificial Sequence
  Description of Artificial Sequence Synthetic oligonucleotide
guaagacuugagaugaucctt

SEQ ID NO: 22 - protein, 8 aa, Artificial Sequence
  Description of Artificial Sequence Synthetic 8xHis tag
HHHHHHHH

(19) United States
(12) Patent Application Publication
(10) Pub. No.: US 2012/0245076 A1
(43) Pub. Date: Sep. 27, 2012

(54) COMPOSITIONS AND METHODS FOR DELIVERING RNAI USING APOE

(75) Inventors: Tomoko Nakayama, Cambridge, MA (US); James Butler, Cambridge, MA (US); Akin Akinc, Cambridge, MA (US)

(73) Assignee: ALNYLAM PHARMACEUTICALS, INC., Cambridge, MA (US)

(21) Appl. No.: 13/394,111

(22) PCT Filed: Sep. 3, 2010
(86) PCT No.: PCT/US10/47834

§ 371 Date: Jun. 13, 2012

Related U.S. Application Data
(60) Provisional application No. 61/239,561, filed on Sep. 3, 2009.
(60) Provisional application No. 61/285,786, filed on Dec. 11, 2009.

(51) Int. Cl. A61K 38/00; A61P 43/00

(52) U.S. Cl. 514/1.1

(57) ABSTRACT

This invention relates to the use of lipoproteins with oligonucleotides, both single and double stranded, and their use in delivering dsRNA for RNA interference. More specifically, the present invention relates to composititons containing oligonucleotides and alipoprotein E, which enables tissue-specific delivery and reduction of target expression.

### CROSS REFERENCE TO RELATED APPLICATIONS

This application claims priority to U.S. Application Ser. No. 61/239,561, filed on Sep. 3, 2009, and U.S. Application Ser. No. 61/285,786, filed on Dec. 11, 2009, the contents of both of which are hereby incorporated by reference in their entirety.

### FIELD OF THE INVENTION

This invention relates to the use of lipoproteins with oligonucleotides, both single and double stranded, and their use in delivering dsRNA for RNA interference. More specifically, the present invention relates to composititons containing oligonucleotides and alipoprotein E, which enables tissue-specific delivery and reduction of target expression.

### BACKGROUND OF THE INVENTION

Recently, double-stranded RNA molecules (dsRNA) have been shown to block gene expression in a highly conserved regulatory mechanism known as RNA interference (RNAi). WO 99/32619 (Fire et al.) discloses the use of a dsRNA of at least 25 nucleotides in length to inhibit the expression of genes in C. elegans. dsRNA has also been shown to degrade target RNA in other organisms, including plants (see, e.g., WO 99/53050, Waterhouse et al.; and WO 99/61631, Heifetz et al.), Drosophila (see, e.g., Yang, D., et al., Curr. Biol. (2000) 10:1191-1200), and mammals (see WO 00/44895, Limmer; and DE 101 00 586.5, Kreutzer et al.). This natural mechanism has now become the focus for the development of a new class of pharmaceutical agents for treating disorders that are caused by the aberrant or unwanted regulation of a gene.

Despite significant advances in the field of RNAi and advances in the treatment of pathological processes, there remains a need for formulations that can selectively and efficiently deliver agents to cells where silencing can then occur.

While delivery of oligonucleotides across plasma membranes in vivo has been achieved using vector-based delivery systems, high-pressure intravenous injections of oligonucleotides and various chemically-modified oligonucleotides, including cholesterol-conjugated, lipid encapsulated and antibody-mediated oligonucleotides, to date, delivery remains the largest obstacle for in vivo oligonucleotide therapeutics.

### SUMMARY OF THE INVENTION

The invention provides compositions containing particles, which contain oligonucleotides in combination with Apolipoprotein E (ApoE), e.g., recombinant ApoE, and methods for inhibiting the expression of a gene in a cell or a mammal. Typically, the particle is substantially devoid of other lipoproteins, such as an ApoA or ApoC. The invention also provides compositions and methods for treating pathological conditions and diseases caused by the expression of a target gene, such as gene whose expression is associated with a lipid-related disease or disorder, such as hyperlipidemia. The oligonucleotides used in combination with ApoE are typically conjugated, for example to a lipophile or otherwise in such a way that permits association into the particles described herein, and can be double stranded or single stranded. The double stranded oligonucleotides featured herein include double-stranded RNA (dsRNA) having an RNA strand (the antisense strand) with a region that is less than 30 nucleotides in length, generally 18-30 nucleotides in length, and having substantial complementarity to at least part of an mRNA transcript of the target gene. In one embodiment, a dsRNA for inhibiting expression of the target gene includes at least two sequences that are complementary to each other. The dsRNA includes a sense strand having a first sequence and an antisense strand having a second sequence. The antisense strand includes a nucleotide sequence that is substantially complementary to at least part of an mRNA encoding target gene, and the region of complementarity is less than 30 nucleotides in length, and at least 18 nucleotides in length. Generally, the dsRNA is 18 to 30, e.g., 19 to 21 nucleotides in length. In one embodiment the strands are independently 18-30 nucleotides.

In like fashion, the single-stranded oligonucleotides associated with ApoE also include a nucleotide sequence that is substantially complementary to at least part of an mRNA encoding target gene, and the region of complementarity is less than 30 nucleotides in length, and at least 15 nucleotides in length. Generally, the single stranded oligonucleotides are 18 to 30, e.g., 19 to 21 nucleotides in length. In one embodiment the strand is 18-30 nucleotides. Single strands having less than 100% complementarity to the target mRNA, RNA or DNA are also embraced by the present invention.

The oligonucleotides featured herein can include naturally occurring nucleotides or can include at least one modified nucleotide, such as a 2′-O-methyl modified nucleotide, a nucleotide having a 5′-phosphorothioate group, and a terminal nucleotide linked to a conjugate group, such as to a cholesteryl derivative, or to a vitamin E group. Alternatively, the modified nucleotide may be chosen from the group of: a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide, a phosphoramidate, and a non-natural base comprising nucleotide.

In some embodiments, the oligonucleotides featured in the invention are stabilized by one or more modifications to avoid degradation of the oligonucleotides. Possible modifications are phosphorothioate units, 2′-O-methyl RNA units, 2′-O-methoxy-ethyl RNA units, peptide nucleic acid units, N3′-P5′ phosphoroamidate DNA units, 2′ fluoro-ribo nucleic acid units, Locked nucleic acid units, morpholino phosphoroamidate nucleic acid units, cyclohexane nucleic acid units, tricyclonucleic acid units, 2′-O-alkylated nucleotide modifications, 2′-Deozy-2′-fluoro modifications, 2,4-difluorotoluoyl modifications, 4′-thio ribose modifications, or boranophospate modifications.

In one embodiment, the particle further comprises a lipid. The lipid can be a phospholipid, which can be of natural origin, such as egg yolk or soybean phospholipids, or synthetic or semisynthetic origin.

In one embodiment, the oligonucleotides featured in the invention are preassembled with an apolipoprotein E, e.g., an ApoE3 isoform of ApoE. It has been surprisingly discovered that when oligonucleotides, either single- or double stranded, are preassembled with high density lipoproteins, both delivery and silencing are effected in tissues in vivo, particularly liver. In one embodiment, 1, 2, 3, 4, 5, 6, or more dsRNAs are incorporated into a reconstituted ApoE, e.g., a reconstituted recombinant ApoE.

In certain embodiments, a particle featured in the invention can include 1 oligonucleotide. In other embodiments, the particle can include about 1 to 3 oligonucleotides, e.g., 2 or 3 oligonucleotides. In another embodiment, the particle can include 3 to 5 oligonucleotides (e.g., 4 or 5 oligonucleotides), 5 to 8 oligonucleotides (e.g., 7 or 8 oligonucleotides), 8 to 10 oligonucleotides, 10 to 15 oligonucleotides, or 15 to 20 or more oligonucleotides. In one embodiment, a particle comprising an oligonucleotide and an ApoE is capable of inhibiting target gene expression to an extent that is 20% greater, 30% greater, 40% greater, 50% greater, 60% greater, or 80% greater or more as compared to when the particle containing the oligonucleotide is contacted with the target gene in the absence of the ApoE.

In one embodiment, the ApoE is an isoform of ApoE, such as an ApoE3, ApoE4, or ApoE2 isoform. In one embodiment, the ApoE has the amino acid sequence of SEQ ID NO:2 (FIG. 9B) or a fragment of SEQ ID NO:2.

In some embodiments, olignucleotides of the particles are conjugated to a lipophile, such as a cholesterol moiety, such as N-(cholesterylcarboxamidocaproyl)-4-hydroxyprolinol (Hyp-C6-Chol (also called “L10”).

In another embodiment, the particle containing an oligonucleotide and an ApoE, e.g., a recombinant ApoE, is less than about 80 nm in diameter. Typically, the particle size is about 5 to 20 nm, e.g., about 6, 8, 10, 12, or 18 nm.

In one aspect, the invention provides a method for selectively targeting and/or delivering an oligonucleotide, such as a dsRNA, to a mammalian tissue, e.g., by contacting the mammal with the oligonucleotide, where the oligonucleotide has been preassembled with an ApoE. In one embodiment, the oligonucleotide is modified with a cholesterol group, and in another embodiment, the dsRNA is selectively targeted and/or delivered to the liver.

In another aspect, the invention provides a pharmaceutical composition for inhibiting the expression of the target gene in an organism, generally a human subject. The composition typically includes apolipoprotein E in combination with one or more dsRNAs, such as one or more exemplary dsRNAs described herein, and a pharmaceutically acceptable carrier or delivery vehicle.

In another aspect, the invention provides methods for treating, preventing or managing pathological processes mediated by a target gene by administering to a patient in need of such treatment, prevention or management a therapeutically or prophylactically effective amount of one or more of the compositions featured herein. In one embodiment, a composition containing particle comprising an oligonucleotide in combination with an ApoE is useful to treat a lipid disorder, or a symptom of a lipid disorder. For example, a composition featured in the invention can include an oligonucleotide that targets a gene involved in cholesterol metabolism, and be administered for treatment of atherosclerosis or hypercholesterolemia or other disorders associated with cholesterol metabolism.

In another aspect, the invention features compositions comprising an oligonucleotide in combination with an ApoE for use in a method described herein, such as for the treatment of a disease or disorder associated with expression (e.g., overexpression) of a target gene. In one embodiment, the disease or disorder is a cancer or a lipid disorder.

### BRIEF DESCRIPTION OF THE FIGURES

FIGS. 1A and 1B are bar graphs illustrating apoB mRNA in liver (FIG. 1A) and jejunum (FIG. 1B) as measured by branched DNA (bDNA) assay.

FIG. 2A is a panel of Western blots showing ApoB 100 and ApoB48 levels following administration of AD5167 to C57BL6, LDLR−/−, and ApoE−/− mice. Bold downward arrows indicate a significant reduction of mRNA levels. 0.5 uL plasma from each animal was loaded per lane.

FIGS. 2B and 2C are bar graphs illustrating ApoB100 (FIG. 8A) and ApoB48 (FIG. 8B) levels as shown in FIG. 2A.

FIGS. 2D-2F are bar graphs illustrating serum cholesterol levels following administration of AD5167 to C57BL6 (FIG. 2D), LDLR−/− (FIG. 2E), and ApoE−/− (FIG. 2F) mice. * means not enough samples.

FIGS. 3A and 3B represent the amino acid sequence of human ApoE with the signal sequence (FIG. 3A, SEQ ID NO:1) and without the signal sequence (FIG. 3B, (SEQ ID NO:2). The amino acid sequence of FIG. 3A is provided at GenBank Accession No. AAB59546.1 (GI:178851) (Oct. 21, 2002).

FIG. 4 is an image of SDS-PAGE showing the expression of ApoE in HEK293 cells and E. coli.

FIGS. 5A and 5B depict a size exclusion analysis of ApoE-rHDL reconstituted with POPC (FIG. 5A) and a Superdex 200 10/30 MW standard curve (FIG. 5B).

FIGS. 6A and 6B depict a size exclusion analysis of ApoE-rHDL reconstituted with DMPC

(FIG. 6A) and a Superdex 200 10/30 MW standard curve (FIG. 6B).

FIG. 7 depicts a size exclusion analysis of AopE-rHDL/AD5167 particles prepared by various EHDL/AD5167 ratios and incubation time.

FIGS. 8A and 8B are bar graphs illustrating ApoB (FIG. 8A) and TTR (FIG. 8B) mRNA levels in a dose response study as measured by branched DNA (bDNA) assay. Bold downward arrows indicate a significant reduction of mRNA levels.

FIG. 9A is a bar graph illustrating ApoB100 protein levels in a dose response study as measured by Western blot analysis.

FIG. 9B is a bar graph illustrating plasma ApoB protein level after administration of AD5167-EHDL particles with ApoE prepared from HEK293 cells or E. coli, or AD5544-EHDL particles, as measured by Western blot analysis.

FIGS. 10A and 10B are bar graphs illustrating apoB mRNA in liver (FIG. 10A) and jejunum (FIG. 10B) after administration of AD5167-EHDL particles with ApoE prepared from HEK293 cells or E. coli, or AD5544-EHDL particles with ApoE prepared from HEK293, as measured by branched DNA (bDNA) assay. Bold downward arrows indicate a significant reduction of mRNA levels.

FIG. 11 is a bar graph illustrating specific knockdown of ApoB mRNA levels in livers of C57BL/6 mice following a single dose (30 mg/kg) administration of rEHDL/chol-siApoB complexes by i.v. (tail vein) injection.

FIG. 12A is a Western blot demonstrating that rEHDL/chol-siApoB complexes administered to mice result in decreased levels of ApoB protein in plasma. Bold downward arrows indicate a significant reduction of mRNA levels. FIG. 12B is a bar graph that provides a quantitative illustration of the significant reduction in plasma ApoB levels resulting from administration of rEHDL/chol-siApoB complexes to mice.

FIG. 12C is a bar graph illustrating decreased plasma cholesterol levels in mice following administration of rEHDL/chol-siApoB complexes.

FIGS. 13A and 13B are bar graphs illustrating specific knockdown of PCSK9 and FVII mRNA, respectively, in liver following administration of 30 mg/kg rEHDL/chol-siPCSK9 and rEHDL/chol-siFVII, respectively.

FIGS. 14A and 14B are bar graphs illustrating specific knockdown of mouse apoB mRNA (FIG. 14A) and human apoB mRNA (FIG. 14B) levels in livers of mice injected with apoB dsRNA (AD5544) or apoB dsRNA complexed with ApoE (rEHDL/AD5544). FIGS. 15A, 15B and 15C are bar graphs illustrating the effect of rEHDL/AD5544 on LDLc, HDLc and total cholesterol levels, respectively, in serum of double transgenic mice.

### DETAILED DESCRIPTION OF THE INVENTION

The invention provides compositions and methods for inhibiting the expression of a target gene in a cell or mammal using single- and/or double-stranded oligonucleotides. In some embodiments, the oligonucleotides are conjugated to one or more lipophiles and preassembled with lipoproteins. This invention is based on the discovery that when lipophilic conjugated oligonucleotides, either single- or double stranded, are preassembled with lipoproteins, both delivery and silencing are effected in tissues in vivo, particularly liver tissue. Oligonucleotides complexed with lipoproteins are also described in U.S. Published Application 2009-0286851, which is incorporated by reference herein in its entirety.

A composition featured in the invention contains a particle that comprises (a) at least one of a single or double stranded oligonucleotide, where said oligonucleotide has been conjugated to a lipophile and (b) an Apolipoprotein E (ApoE), such as a recombinant ApoE. The particle is typically substantially devoid of other apolipoproteins. By “substantially devoid” is meant that the apolipoprotein component of the particle is >98%, >99%, or >99.5% or more ApoE or derived from ApoE (e.g., fragments of ApoE). As used herein, a particle comprising an oligonucleotide and an ApoE is understood to be a particle comprising an oligonucleotide complexed with an ApoE, or pre-assempled with an ApoE, or formulated with an ApoE. These terms and expressions are used interchangeably herein.

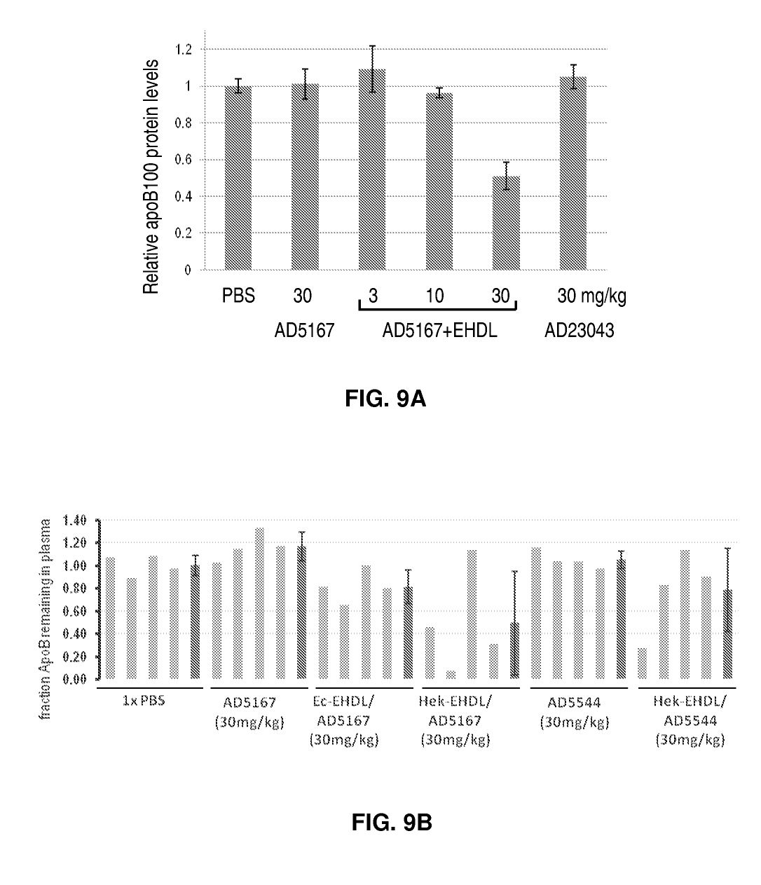

ApoE is a 299 amino acid polypeptide and a component of very low density lipoproteins (VLDLs), chylomicron, chylomicron remnants, intermediate density lipoproteins (IDLs) and a subclass of high density lipoproteins (HDL). The three major isoforms of ApoE are ApoE3, ApoE4 and ApoE2. ApoE3 carries a cysteine at position 112 and an arginine at position 158, and is the most common form (FIG. 9B). ApoE4 carries a cysteine at positions 112 and 158, and is associated with type III hyperlipoproteinemia. ApoE2 carries an arginine at position 112 and an arginine at position 158, and is associated with cardiovascular and neurodenerative diseases.

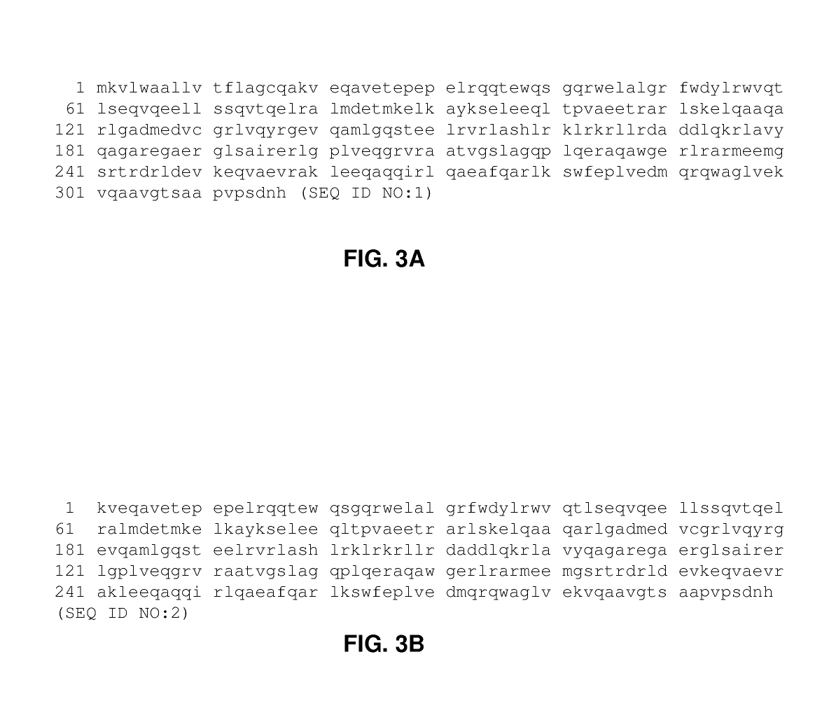

The amino acid sequence of human preapolipoprotein E (which includes an 18 amino acid signal sequence), is provided at GenBank Accession No. AAB59546.1 (GI:178851) (FIG. 3A, SEQ ID NO:1). The amino acid sequence of the mature human apolipoprotein E (which does not include the signal sequence) is provided at FIG. 3B.

The arginine rich region of ApoE at amino acids positions 136-158 (SEQ ID NO:2, FIG. 9B) interacts with the LDL receptor (LDLR), and the C terminal domain at amino acid positions 216-299 binds lipoprotein particles. ApoE also interacts with glucosylaminoglycans including heparin.

A recombinant ApoE is made by methods known in the art, such as in a bacterial cell, e.g., an E. coli cell, or a mammalian cell, such as a human cell. The ApoE can be glycosylated or unglycosylated.

The invention provides compositions and methods for treating pathological conditions and diseases, such as lipid diseases or disorders. The oligonucleotide component is single stranded or double stranded and can direct the sequence-specific degradation of mRNA through the antisense mechanism known as RNA interference (RNAi). In some embodiments, the oligonucleotide is an saRNA, such as for use in RNA activation. RNA activation is described, e.g., in WO2006/113246, filed Apr. 11, 2006, which is incorporated by reference herein in its entirety.

The oligonucleotides of the compositions featured herein include dsRNAs, which comprise an RNA strand (the antisense strand) having a region which is less than 30 nucleotides in length, generally 18-30 nucleotides in length, specifically 21-23 nucleotides in length and is substantially complementary to at least part of an mRNA transcript of the target gene. In one embodiment the oligonucleotides are specifically between 18-30 nucleotides in length, encompassing those of 18, 19, 20, 21, 22, 23, 24, 25, 26, 27, 28, 29 or 30 nucleotides. The use of these dsRNAs enables the targeted degradation of mRNAs of genes that are implicated in replication and or maintenance of disease states, e.g. cancer, in mammals. Very low dosages of formulated dsRNAs with lipoproteins in particular can specifically and efficiently mediate RNAi, resulting in significant inhibition of expression of the target gene. The methods and compositions containing the formulated dsRNAs are useful for treating pathological processes mediated by target gene expression.

The pharmaceutical compositions featured in the invention include a dsRNA having an antisense strand comprising a region of complementarity which is less than 30 nucleotides in length, generally 18-30 nucleotides in length, and is substantially complementary to at least part of an RNA transcript of the target gene, optionally with a pharmaceutically acceptable carrier.

Lipoproteins contain both proteins and lipids, and are classified as follows (listed from larger and less dense to smaller and more dense): chylomicrons, very low density lipoproteins (VLDL), intermediate density lipoproteins (IDL), low density lipoproteins (LDL) and high density lipoproteins (HDL). Lipoproteins are larger and less dense, if they consist of more fat than protein. Chylomicrons typically carry triglycerides (fat) from the intestines to the liver, skeletal muscle, and to adipose tissue; VLDL typically carry (newly synthesised) triacylglycerol from the liver to adipose tissue; IDL are intermediate between VLDL and LDL and are not usually detectable in the blood; LDL typically carry cholesterol from the liver to cells of the body; and HDL typically collect cholesterol from the body's tissues, and bring it back to the liver.

Apolipoproteins are proteins that bind to lipids to form lipoproteins. The lipid components of lipoproteins are not soluble in water. However, apolipoproteins and other amphipathic molecules (such as phospholipids) can surround the lipids, creating the lipoprotein particle that is itself water-soluble, and can thus be carried through water-based circulation (i.e., blood, lymph).

Reconstituted Lipoproteins

Methods of producing reconstituted lipoproteins have been described in scientific literature, especially for apolipoproteins A-I, A-II, A-IV, apoC and ApoE (A. Jonas, Methods in Enzymology 128, 553-582 (1986). The most frequent lipid used for reconstitution is phosphatidylcholine, extracted either from eggs or soybeans. Other phospholipids are also used, also lipids such as triglycerides or cholesterol. For reconstitution, the lipids are first dissolved in an organic solvent, which is subsequently evaporated under nitrogen. In this method the lipid is bound in a thin film to a glass wall. Afterwards the apolipoproteins and a detergent, normally sodium cholate, are added and mixed. The added sodium cholate causes a dispersion of the lipid. After a suitable incubation period, the mixture is dialyzed against large quantities of buffer for a longer period of time; the sodium cholate is thereby removed for the most part, and at the same time lipids and apolipoproteins spontaneously form themselves into lipoproteins or so-called reconstituted lipoproteins.

As alternatives to dialysis, hydrophobic adsorbents are available which can adsorb detergents (Bio-Beads SM-2, Bio Rad; Amberlite XAD-2, Rohm & Haas) (E. A. Bonomo, J. B. Swaney, J. Lipid Res., 29, 380-384 (1988)), or the detergent can be removed by means of gel chromatography (Sephadex G-25, Pharmacia). Lipoproteins can also be produced without detergents, for example through incubation of an aqueous suspension of a suitable lipid with apolipoproteins, the addition of lipid which was dissolved in an organic solvent, to apolipoproteins, with or without additional heating of this mixture, or through treatment of an apoA-1-lipid-mixture with ultrasound. With these methods, starting, for example, with apoA-I and phosphatidyl choline, disk-shaped particles can be obtained which correspond to lipoproteins in their nascent state. Discoidal reconstituted high density lipoproteins (rHDL) can also be prepared from ApoE and phospholipids. Normally, following the incubation, unbound apolipoproteins and free lipid are separated by means of centrifugation or gel chromatography in order to isolate the homogeneous, reconstituted lipoproteins particles. U.S. Pat. No. 5,128,318 describes a method of producing rHDL wherein phosphatidyl choline is dissolved in a solution with the aid of an organic solvent.

There are many disclosures of synthetic HDL-particles in the literature which refer to their capacity in picking up and removing undesired lipid material in the blood stream and from the blood vessels thus making them potentially useful in therapy for treating atherosclerosis by depleting cholesterol from arterial plaques and for removing lipid soluble toxins such as endotoxins.

In Experimental Lung Res. 1984, Vol. 6, pp. 255-270: A Jonas, experimental conditions of forming complexes of the partially hydrophobic apolipoproteins and phospholipids are described in detail. It was found that, by contacting apolipoproteins with preformed phosphatidyl choline vesicles, lipid particles were spontaneously formed which could be used as analogs of HDL-particles. By mixing phosphatidyl choline and bile acids to a miscellar dispersion and contacting the resultant mixture with apolipoproteins specifically shaped, discoidal and thermodynamically stable lipid particles were formed by means of a dialysis method, subsequently called the “cholate-dialysis method.”

U.S. Pat. No. 4,643,988 to Research Corporation describes synthetic peptides useful in treatment of atherosclerosis with an improved amphiphatic helix and an ability to spontaneously form stable discoidal lipid particles with phospholipids which resemble native HDL-complexes. The lipid particles can be formed by contacting vesicles of phosphatidyl choline made by sonication. However, such a production method including sonication is suitable only for smaller batches of lipid particles and not for large scale pharmaceutical production.

U.S. Pat. No. 5,128,318 to Rogosin Institute describes the production of reconstituted lipoprotein containing particles (HDL-particles) from plasma derived apolipoproteins which are processed to synthetic particles for parenteral administration with the addition of cholate and egg yolk phosphatidyl choline. A similar method is also disclosed in the Japanese patent application JP 61-152632 to Daiichi Seiyaku KK.

Also in WO 87/02062 to Biotechn. Res. Partners LTD, it is disclosed how to obtain a stabilized formulation by incubating a solution of recombinantly produced lipid binding protein, such as human apolipoprotein, with a conventional lipid emulsion for parenteral nutrition.

The article by G. Franceschini et al. in J. Biol. Chem., 1985, Vol. 260 (30), pp. 16231-25 considers the spontaneous formation of lipid particles between apolipoprotein A-I and phosphatidyl choline. In this article, it is also revealed that Apo-IM (Milano), the variant of apolipoprotein A-I carried by individuals shown to have a very low prevalence of atherosclerosis, has a higher affinity (association rate) to dimyristoyl phosphatidyl choline (DMPC) than regular Apo A-I. It is suggested that the mutant Apo A-IM has a slightly higher exposure of hydrophobic residues which may contribute both an accelerated catabolism and an improved tissue lipid uptake capacity of such Apo A-IM/DMPC particles.

The Canadian patent application CA 2138925 to the Swiss Red Cross discloses an improved, more industrially applicable, method of producing synthetic rHDL particles from purified serum apolipoproteins and phospholipids which avoids organic solvents while resulting in less unbound, free non-complexed phospholipids (i.e. a higher yield of lipoprotein particles). Herein, it is suggested to mix an aqueous solution of apolipoproteins with an aqueous solution of phospholipid and bile acids, whereupon the resultant mixture is incubated and protein-phospholipid particles are spontaneously formed when bile acids are removed from phospholipid/bile acid micelles with diafiltration.

Phospholipids which can be of natural origin, such as egg yolk or soybean phospholipids, or synthetic or semisynthetic origin. The phospholipids can be partially purified or fractionated to comprise pure fractions or mixtures of phosphatidyl cholines, phosphatidyl ethanolamines, phosphatidyl inositols, phosphatidic acids, phosphatidyl serines, sphingomyelin or phosphatidyl glycerols. In certain embodiments, phospholipids with defined fatty acid radicals, such as dimyristoyl phosphatidyl choline (DMPC), dioleoylphosphatidylethanolamine (DOPE), palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), -phosphatidylethanolamine (POPE), dioleoyl-phosphatidylethanolamine 4-(N-maleimidomethyl)-cyclohexane-1-carboxylate (DOPE-mal), and combinations thereof, and the like phosphatidyl cholines with defined acyl groups selected from naturally occurring fatty acids, generally having 8 to 22 carbon atoms, will be selected. According to a specific embodiment, phosphatidyl cholines having only saturated fatty acid residues between 14 and 18 carbon atoms will be used, and of those dipalmitoyl phosphatidyl choline will be typical.

Phospholipids suitable for reconstitution with lipoproteins such as ApoE lipoproteins include, e.g., phosphatidylcholine, phosphatidylglycerol, lecithin, b, g-dipalmitoyl-a-lecithin, sphingomyelin, phosphatidylserine, phosphatidic acid, N-(2,3-di(9-(Z)-octadecenyloxy))-prop-1-yl-N,N,N-trimethylammonium chloride, phosphatidylethanolamine, lysolecithin, lysophosphatidylethanolamine, phosphatidylinositol, cephalin, cardiolipin, cerebrosides, dicetylphosphate, dioleoylphosphatidylcholine, dipalmitoylphosphatidylcholine, dipalmitoylphosphatidylglycerol, dioleoylphosphatidylglycerol, palmitoyl-oleoyl-phosphatidylcholine, di-stearoyl-phosphatidylcholine, stearoyl-palmitoyl-phosphatidylcholine, di-palmitoyl-phosphatidylethanolamine, di-stearoyl-phosphatidylethanolamine, di-myrstoyl-phosphatidylserine, di-oleyl-phosphatidylcholine, and the like. Non-phosphorus containing lipids may also be used in the liposomes of the compositions featured in the invention. These include, e.g., stearylamine, docecylamine, acetyl palmitate, fatty acid amides, and the like.

Besides the amphiphilic agent, the lipid agent may comprise, in various amounts at least one nonpolar component which can be selected among pharmaceutical acceptable oils (triglycerides) exemplified by the commonly employed vegetabilic oils such as soybean oil, safflower oil, olive oil, sesame oil, borage oil, castor oil and cottonseed oil or oils from other sources like mineral oils or marine oils including hydrogenated and/or fractionated triglycerides from such sources. Also medium chain triglycerides (MCT-oils, e.g. Miglyol®), and various synthetic or semisynthetic mono-, di- or triglycerides, such as the defined nonpolar lipids disclosed in WO 92/05571 may be used in the present invention as well as acetylated monoglycerides, or alkyl esters of fatty acids, such isopropyl myristate, ethyl oleate (see EP 0 353 267) or fatty acid alcohols, such as oleyl alcohol, cetyl alcohol or various nonpolar derivatives of cholesterol, such as cholesterol esters.

One or more complementary surface active agent can be added to the composition featured in this invention, for example as complements to the characteristics of amphiphilic agent or to improve its lipid particle stabilizing capacity or enable an improved solubilization of the protein. Such complementary agents can be pharmaceutically acceptable non-ionic surfactants, such as alkylene oxide derivatives of an organic compound which contains one or more hydroxylic groups. For example, ethoxylated and/or propoxylated alcohol or ester compounds or mixtures thereof are commonly available and are well known as such complements to those skilled in the art. Examples of such compounds are esters of sorbitol and fatty acids, such as sorbitan monopalmitate or sorbitan monopalmitate, oily sucrose esters, polyoxyethylene sorbitane fatty acid esters, polyoxyethylene sorbitol fatty acid esters, polyoxyethylene fatty acid esters, polyoxyethylene alkyl ethers, polyoxyethylene sterol ethers, polyoxyethylene-polypropoxy alkyl ethers, block polymers and cethyl ether, as well as polyoxyethylene castor oil or hydrogenated castor oil derivatives and polyglycerine fatty acid esters. Suitable non-ionic surfactants, include, but are not limited to various grades of Pluronic®, Poloxamer®, Span®, Tween®, Polysorbate®, Tyloxapol®, Emulphor® or Cremophor® and the like. The complementary surface active agents may also be of an ionic nature, such as bile duct agents, cholic acid or deoxycholic their salts and derivatives or free fatty acids, such as oleic acid, linoleic acid and others. Other ionic surface active agents are found among cationic lipids like C10-C24: alkylamines or alkanolamine and cationic cholesterol esters.

Also other pharmacologically acceptable components can be added to the lipid agent when desired, such as antioxidants (exemplified by alpha-tocopherol) and solubilization adjuvants (exemplified by benzylalcohol).

### Oligonucleotides

### Double-Stranded Oligonucleotides

In one embodiment, the invention provides double-stranded ribonucleic acid (dsRNA) molecules for inhibiting the expression of the target gene (alone or incombinaton with a second dsRNA for inhibiting the expression of a second target gene) in a cell or mammal, wherein the dsRNA comprises an antisense strand comprising a region of complementarity which is complementary to at least a part of an mRNA formed in the expression of the target gene, and wherein the region of complementarity is less than 30 nucleotides in length, generally 19-24 nucleotides in length, e.g., 19 to 21 nucleotides in length. In some embodiments, the dsRNA is from about 10 to about 15 nucleotides, and in other embodiments the dsRNA is from about 25 to about 30 nucleotides in length. In another embodiment, the dsRNA is at least 15 nucleotides in length. The dsRNA, upon contact with a cell expressing said target gene, inhibits the expression of said target gene. The dsRNA includes two RNA strands that are sufficiently complementary to hybridize to form a duplex structure. Generally, the duplex structure is between 15 and 30, more generally between 18 and 30, yet more generally between 19 and 24, and most generally between 19 and 21 base pairs in length. In certain embodiments, longer dsRNAs of between 18 and 30 base pairs in length are typical. Similarly, the region of complementarity to the target sequence is generally between 15 and 30, more generally between 18 and 25, yet more generally between 19 and 24, and most generally between 19 and 21 nucleotides in length. In some embodiments, the dsRNA is between 10 and 15 nucleotides in length, and in other embodiments, the dsRNA is between 25 and 30 nucleotides in length. The dsRNA may further include one or more single-stranded nucleotide overhang(s).

The dsRNA can be synthesized by standard methods known in the art as further discussed below, e.g., by use of an automated DNA synthesizer, such as are commercially available from, for example, Biosearch, Applied Biosystems, Inc. In one embodiment, the target gene is a human target gene.

The skilled person is well aware that dsRNAs comprising a duplex structure of between 20 and 23, but specifically 21, base pairs have been hailed as particularly effective in inducing RNA interference (Elbashir et al., EMBO 2001, 20:6877-6888). However, others have found that shorter or longer dsRNAs can be effective as well. In the embodiments described above the dsRNAs can include at least one strand of a length of minimally 21 nucleotides. It can be reasonably expected that shorter dsRNAs comprising a known sequence minus only a few nucleotides on one or both ends may be similarly effective as compared to the dsRNAs of the lengths described above. Hence, dsRNAs comprising a partial sequence of at least 15, 16, 17, 18, 19, 20, or more contiguous nucleotides, and differing in their ability to inhibit the expression of the target gene in a FACS assay as described herein below by not more than 5, 10, 15, 20, 25, or 30% inhibition from a dsRNA comprising the full sequence, are contemplated by the invention. Further dsRNAs that cleave within the target sequence can readily be made using the target gene sequence and the target sequence provided.

The present invention further features dsRNAs that target within the sequence targeted by one of the agents described herein. A second dsRNA is understood to target within the sequence of a first dsRNA, if the second dsRNA cleaves the message anywhere within the mRNA that is complementary to the antisense strand of the first dsRNA. Such a second dsRNA will generally consist of at least 15 contiguous nucleotides coupled to additional nucleotide sequences taken from the region contiguous to the selected sequence in the target gene.

The dsRNA featured in the invention can contain one or more mismatches to the target sequence. In one embodiment, the dsRNA contains no more than 3 mismatches. If the antisense strand of the dsRNA contains mismatches to a target sequence, then the area of mismatch is typically not be located in the center of the region of complementarity. If the antisense strand of the dsRNA contains mismatches to the target sequence, then the mismatch is typically restricted to 5 nucleotides from either end, for example 5, 4, 3, 2, or 1 nucleotide from either the 5′ or 3′ end of the region of complementarity. For example, for a 23 nucleotide dsRNA strand which is complementary to a region of the target gene, the dsRNA generally does not contain any mismatch within the central 13 nucleotides. The methods described within the invention can be used to determine whether a dsRNA containing a mismatch to a target sequence is effective in inhibiting the expression of the target gene. Consideration of the efficacy of dsRNAs with mismatches in inhibiting expression of the target gene is important, especially if the particular region of complementarity in the target gene is known to have polymorphic sequence variation within the population.

In one embodiment, at least one end of the dsRNA has a single-stranded nucleotide overhang of 1 to 4, generally 1 or 2 nucleotides. dsRNAs having at least one nucleotide overhang have unexpectedly superior inhibitory properties than their blunt-ended counterparts. Moreover, the present inventors have discovered that the presence of only one nucleotide overhang strengthens the interference activity of the dsRNA, without affecting its overall stability. dsRNA having only one overhang has proven particularly stable and effective in vivo, as well as in a variety of cells, cell culture mediums, blood, and serum. Generally, the single-stranded overhang is located at the 3′-terminal end of the antisense strand or, alternatively, at the 3′-terminal end of the sense strand. The dsRNA may also have a blunt end, generally located at the 5′-end of the antisense strand. Such dsRNAs have improved stability and inhibitory activity, thus allowing administration at low dosages, i.e., less than 5 mg/kg body weight of the recipient per day. In one embodiment, the antisense strand of the dsRNA has a 1-10 nucleotide overhang at the 3′ end and/or the 5′ end. In another embodiment, the sense strand of the dsRNA has a 1-10 nucleotide overhang at the 3′ end and/or the 5′ end. In yet another embodiment, one or more of the nucleotides in the overhang is replaced with a nucleoside thiophosphate.

In yet another embodiment, the dsRNA is chemically modified to enhance stability. The nucleic acids featured in the invention may be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference. Specific examples of typical dsRNA compounds useful in this invention include dsRNAs containing modified backbones or no natural internucleoside linkages. As defined in this specification, dsRNAs having modified backbones include those that retain a phosphorus atom in the backbone and those that do not have a phosphorus atom in the backbone. For the purposes of this specification, and as sometimes referenced in the art, modified dsRNAs that do not have a phosphorus atom in their internucleoside backbone can also be considered to be oligonucleosides.

Typical modified dsRNA backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; and 5,625,050, each of which is herein incorporated by reference

Modified dsRNA backbones that do not include a phosphorus atom have backbones that are formed by short chain alkyl or cycloalkyl internucleoside linkages, mixed heteroatoms and alkyl or cycloalkyl internucleoside linkages, or ore or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, each of which is herein incorporated by reference.

In other dsRNA mimetics, both the sugar and the internucleoside linkage, i.e., the backbone, of the nucleotide units are replaced with novel groups. The base units are maintained for hybridization with an appropriate nucleic acid target compound. One such oligomeric compound, an dsRNA mimetic that has been shown to have excellent hybridization properties, is referred to as a peptide nucleic acid (PNA). In PNA compounds, the sugar backbone of an dsRNA is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

A typical dsRNA will have a phosphorothioate backbone and oligonucleosides with a heteroatom backbone, and in particular —CH₂NHCH₂—, —CH₂N(CH₃)OCH₂ [known as a methylene (methylimino) or MMI backbone], —CH₂ON(CH₃)—CH₂—, —CH₂N(CH₃)N(CH₃)CH₂— and —N(CH₃)—CH₂—CH₂— [wherein the native phosphodiester backbone is represented as —OPOCH₂—] of the above-referenced U.S. Pat. No. 5,489,677, and an amide backbone of the above-referenced U.S. Pat. No. 5,602,240. In some embodiments, a dsRNA will have a morpholino backbone structures of the above-referenced U.S. Pat. No. 5,034,506.

Modified dsRNAs may also contain one or more substituted sugar moieties. Typical dsRNAs include one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Other embodiments include, e.g., O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other typical dsRNAs include one of the following at the 2′ position: C1 to C10 lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an dsRNA, or a group for improving the pharmacodynamic properties of an dsRNA, and other substituents having similar properties. One modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxy-alkoxy group. Another modification includes 2′-dimethylaminooxyethoxy, i.e., a O(CH2)2ON(CH₃)₂ group, also known as 2′-DMAOE, as described in the examples below, and 2′-dimethylaminoethoxyethoxy (also known in the art as 2′-O-dimethylaminoethoxyethyl or 2′-DMAEOE), i.e., 2′-OCH₂OCH₂N(CH₂)₂, also described in the examples below.

Other modifications include 2′-methoxy (2′-OCH₃), 2′-aminopropoxy (2′-OCH₂CH₂CH₂NH₂) and 2′-fluoro (2′-F). Similar modifications may also be made at other positions on the dsRNA, particularly the 3′ position of the sugar on the 3′ terminal nucleotide or in 2′-5′ linked dsRNAs and the 5′ position of 5′ terminal nucleotide. DsRNAs may also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application, and each of which is herein incorporated by reference in its entirety.

DsRNAs may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in The Concise Encyclopedia Of Polymer Science And Engineering, pages 858-859, Kroschwitz, J. L, ed. John Wiley & Sons, 1990, these disclosed by Englisch et al., Angewandte Chemie, International Edition, 1991, 30, 613, and those disclosed by Sanghvi, Y. S., Chapter 15, DsRNA Research and Applications, pages 289-302, Crooke, S. T. and Lebleu, B., Ed., CRC Press, 1993. Certain of these nucleobases are particularly useful for increasing the binding affinity of the oligomeric compounds featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., DsRNA Research and Applications, CRC Press, Boca Raton, 1993, pp. 276-278) and represent typical base substitutions, particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, each of which is herein incorporated by reference, and U.S. Pat. No. 5,750,692, also herein incorporated by reference.

Another typical modification of dsRNAs involves chemically linking to the dsRNA one or more moieties or conjugates which enhance the activity, cellular distribution or cellular uptake of the dsRNA. Such moieties include but are not limited to lipid moieties such as a cholesterol moiety (Letsinger et al., Proc. Natl. Acid. Sci. USA, 199, 86, 6553-6556), cholic acid (Manoharan et al., Biorg. Med. Chem. Let., 1994 4 1053-1060), a thioether, e.g., beryl-S-tritylthiol (Manoharan et al., Ann. N.Y. Acad. Sci., 1992, 660, 306-309; Manoharan et al., Biorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-Hphosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyloxycholesterol moiety (Crooke et al., J. Pharmacol. Exp. Ther., 1996, 277, 923-937).

Representative U.S. patents that teach the preparation of such dsRNA conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

It is not necessary for all positions in a given compound to be uniformly modified, and in fact more than one of the aforementioned modifications may be incorporated in a single compound or even at a single nucleoside within an dsRNA. The present invention also includes dsRNA compounds which are chimeric compounds. “Chimeric” dsRNA compounds or “chimeras,” in the context of this invention, are dsRNA compounds, particularly dsRNAs, which contain two or more chemically distinct regions, each made up of at least one monomer unit, i.e., a nucleotide in the case of an dsRNA compound. These dsRNAs typically contain at least one region wherein the dsRNA is modified so as to confer upon the dsRNA increased resistance to nuclease degradation, increased cellular uptake, and/or increased binding affinity for the target nucleic acid. An additional region of the dsRNA may serve as a substrate for enzymes capable of cleaving RNA:DNA or RNA:RNA hybrids. By way of example, RNase H is a cellular endonuclease which cleaves the RNA strand of an RNA:DNAduplex. Activation of RNase H, therefore, results in cleavage of the RNA target, thereby greatly enhancing the efficiency of dsRNA inhibition of gene expression. Consequently, comparable results can often be obtained with shorter dsRNAs when chimeric dsRNAs are used, compared to phosphorothioate deoxydsRNAs hybridizing to the same target region. Cleavage of the RNA target can be routinely detected by gel electrophoresis and, if necessary, associated nucleic acid hybridization techniques known in the art.

In certain instances, the dsRNA may be modified by a non-ligand group. A number of non-ligand molecules have been conjugated to dsRNAs in order to enhance the activity, cellular distribution or cellular uptake of the dsRNA, and procedures for performing such conjugations are available in the scientific literature. Such non-ligand moieties have included lipid moieties, such as cholesterol (Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-5-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Representative United States patents that teach the preparation of such dsRNA conjugates have been listed above. Typical conjugation protocols involve the synthesis of dsRNAs bearing an aminolinker at one or more positions of the sequence. The amino group is then reacted with the molecule being conjugated using appropriate coupling or activating reagents. The conjugation reaction may be performed either with the dsRNA still bound to the solid support or following cleavage of the dsRNA in solution phase. Purification of the dsRNA conjugate by HPLC typically affords the pure conjugate.

In some embodiments, an oligonucleotide described herein is covalently bound to a lipophilic ligand. Exemplary lipophilic ligands include cholesterol; bile acids; and fatty acids (e.g., lithocholic-oleyl acid, lauroyl acid, docosnyl acid, stearoyl acid, palmitoyl acid, myristoyl acid, oleoyl acid, or linoleoyl acid). The lipophilic ligand can be bound to the oligonucleotide directly or indirectly, for example, via a tether such as a tether that includes a cleavable linking group. In some embodiments, the lipophilic ligand is bound to the oligoneucleotide via a position on the oligonucleotide wherein a ribose of the oligonucleotide has been replaced, for example, by a monomer such as a pyrrolidine monomer.

Exemplary oligonucleotides covalently bound to a lipophilic moiety include the following structure of formula (I), incorporated into the oligonucleotide (e.g., an oligonucleotide described herein):

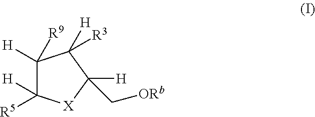

wherein:

### X is N(CO)R⁷, or NR⁷;

each of R³, R⁵ and R⁹, is, independently, H, OH, OR^{a}, OR^{b};
R⁷ is C₁-C₂₀ alkyl substituted with NR^{c}R^{d} or NHC(O)R^{d};

### R^{a} is:

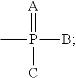

### R^{b} is

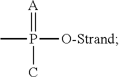

each of A and C is, independently, O or S;

### B is OH, O—, or

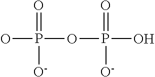

R^{c} is H or C₁-C₆ alkyl; and
R^{d} is a lipophilic ligand, including, for example, cholesterol; a bile acid; or a fatty acid (e.g., lithocholic-oleyl acid, lauroyl acid, docosnyl acid, stearoyl acid, palmitoyl acid, myristoyl acid, oleoyl acid, or linoleoyl acid). The lipophilic ligand, in some embodiments, can be furether tethered to a carbohydrate radical. Other exemplary monomers, which can be incorporated into an oligonucleotide described herein and covalently bound to a lipophilic moiety are described, for example, in US 2005/0107325, which is incorporated by reference herein in its entirety.

### Single-Stranded Oligonucleotides

Single stranded oliogonucleotides, including those described and/or identified as microRNAs or mirs which may be used as targets or may serve as a template for the design of oligonucleotides are taught in, for example, Esau, et al. US Publication 20050261218 (USSN: 10/909,125) entitled “Oligomeric compounds and compositions for use in modulation small non-coding RNAs” the entire contents of which is incorporated herein by reference. It will be appreciated by one of skill in the art that any chemical modifications or variations which apply to the double stranded oligonucleotides described above, also apply to single stranded oligonucleotides. As such, said description has not been repeated here.

The stoichiometry of oligonucleotide to lipoprotein may be 1:1. Alternatively the stoichiometry may be 1:many, many:1 or many:many, where many is greater than 2.

In one embodiment, the composition includes ApoE to oligonucleotide in a ratio of at least 1:1, e.g., about 1:3, about 1:8, about 1:10, about 1:15, or about 1:20.

### DEFINITIONS

For convenience, the meaning of certain terms and phrases used in the specification, examples, and appended claims, are provided below. If there is an apparent discrepancy between the usage of a term in other parts of this specification and its definition provided in this section, the definition in this section shall prevail.

The term “preassembled” is intended to encompass standard mixing of the agents of a composition. The term is also intended to embrace formation of complex between the agents of a composition. Complex can be via chemical interaction, such as, e.g., covalent, ionic, or secondary bonding (e.g., hydrogen bonding), and the like, or via physical interaction, such as, e.g., encapsulation, entrapment, and the like. The complex is formed prior to administration to a patient.

The phrase “RNA interference” and the term “RNAi” are synonymous and refer to the process by which a single, double, or tripartite molecule (e.g. an siRNA, a dsRNA, an shRNA, an miRNA, a piRNA) exerts an effect on a biological process by interacting with one or more components of the RNAi pathway including but not limited to Drosha, RISC, Dicer, etc. The process includes, but is not limited to, gene silencing by degrading mRNA, attenuating translation, interactions with tRNA, rRNA, hnRNA, cDNA and genomic DNA, inhibition of as well as methylation of DNA with ancillary proteins. In addition, molecules that modulate RNAi (e.g. siRNA, piRNA, or miRNA inhibitors) are included in the list of molecules that enhance the RNAi pathway (Tomari, Y. et al. Genes Dev. 2005, 19(5):517-29). The terms siRNA and dsRNA are used interchangeably herein.

“G,” “C,” “A,” “T” and “U” each generally stand for a nucleotide that contains guanine, cytosine, adenine, thymidine and uracil as a base, respectively. However, it will be understood that the term “ribonucleotide” or “nucleotide” can also refer to a modified nucleotide, as further detailed below, or a surrogate replacement moiety. The skilled person is well aware that guanine, cytosine, adenine, thymidine and uracil may be replaced by other moieties without substantially altering the base pairing properties of an oligonucleotide comprising a nucleotide bearing such replacement moiety. For example, without limitation, a nucleotide comprising inosine as its base may base pair with nucleotides containing adenine, cytosine, or uracil. Hence, nucleotides containing uracil, guanine, or adenine may be replaced in the nucleotide sequence by a nucleotide containing, for example, inosine. In another example, adenine and cytosine anywhere in the oligonucleotide can be replaced with guanine and uracil, respectively to form G-U Wobble base pairing with the target mRNA. Sequences comprising such replacement moieties are exemplary embodiments.

As used herein, “target sequence” refers to a contiguous portion of the nucleotide sequence of an mRNA molecule formed during the transcription of the target gene, including mRNA that is a product of RNA processing of a primary transcription product. Target sequences may further include RNA precursors, either pri or pre-microRNA, or DNA which encodes the mRNA.

As used herein, the term “strand comprising a sequence” refers to an oligonucleotide comprising a chain of nucleotides that is described by the sequence referred to using the standard nucleotide nomenclature.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions may include: 400 mM NaCl, 40 mM PIPES pH 6.4, 1 mM EDTA, 50° C. or 70° C. for 12-16 hours followed by washing. Other conditions, such as physiologically relevant conditions as may be encountered inside an organism, can apply. The skilled person will be able to determine the set of conditions most appropriate for a test of complementarity of two sequences in accordance with the ultimate application of the hybridized nucleotides.

This includes base-pairing of the oligonucleotide or polynucleotide comprising the first nucleotide sequence to the oligonucleotide or polynucleotide comprising the second nucleotide sequence over the entire length of the first and second nucleotide sequence. Such sequences can be referred to as “fully complementary” with respect to each other herein. However, where a first sequence is referred to as “substantially complementary” with respect to a second sequence herein, the two sequences can be fully complementary, or they may form one or more, but generally not more than 4, 3 or 2 mismatched base pairs upon hybridization, while retaining the ability to hybridize under the conditions most relevant to their ultimate application. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, may yet be referred to as “fully complementary.”

“Complementary” sequences, as used herein, may also include, or be formed entirely from, non-Watson-Crick base pairs and/or base pairs formed from non-natural and modified nucleotides, in as far as the above requirements with respect to their ability to hybridize are fulfilled. Such non-Watson-Crick base pairs includes, but not limited to, G:U Wobble or Hoogstein base pairing.

The terms “complementary”, “fully complementary” and “substantially complementary” herein may be used with respect to the base matching between the sense strand and the antisense strand of a dsRNA, or between the antisense strand of a dsRNA and a target sequence, as will be understood from the context of their use.

As used herein, a polynucleotide which is “substantially complementary to at least part of” a messenger RNA (mRNA) refers to a polynucleotide which is substantially complementary to a contiguous portion of the mRNA of interest (e.g., encoding target gene). For example, a polynucleotide is complementary to at least a part of a target gene mRNA if the sequence is substantially complementary to a non-interrupted portion of a mRNA encoding target gene.

As used herein the term “oligonucleotide” embraces both single and double stranded polynucleotides.

The term “double-stranded RNA” or “dsRNA”, as used herein, refers to a complex of ribonucleic acid molecules, having a duplex structure comprising two anti-parallel and substantially complementary, as defined above, nucleic acid strands. The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′ end of the respective other strand forming the duplex structure, the connecting RNA chain is referred to as a “hairpin loop”. Where the two strands are connected covalently by means other than an uninterrupted chain of nucleotides between the 3′-end of one strand and the 5′ end of the respective other strand forming the duplex structure, the connecting structure is referred to as a “linker.” The RNA strands may have the same or a different number of nucleotides. The maximum number of base pairs is the number of nucleotides in the shortest strand of the dsRNA minus any overhangs that are present in the duplex. In addition to the duplex structure, a dsRNA may comprise one or more nucleotide overhangs.

As used herein, a “nucleotide overhang” refers to the unpaired nucleotide or nucleotides that protrude from the duplex structure of a dsRNA when a 3′-end of one strand of the dsRNA extends beyond the 5′-end of the other strand, or vice versa. “Blunt” or “blunt end” means that there are no unpaired nucleotides at that end of the dsRNA, i.e., no nucleotide overhang. A “blunt ended” dsRNA is a dsRNA that is double-stranded over its entire length, i.e., no nucleotide overhang at either end of the molecule.

The term “antisense strand” refers to the strand of a dsRNA which includes a region that is substantially complementary to a target sequence. As used herein, the term “region of complementarity” refers to the region on the antisense strand that is substantially complementary to a sequence, for example a target sequence, as defined herein. Where the region of complementarity is not fully complementary to the target sequence, the mismatches may be in the internal or terminal regions of the molecule. Generally, the most tolerated mismatches are in the terminal regions, e.g., within 6, 5, 4, 3, or 2 nucleotides of the 5′ and/or 3′ terminus.

The term “sense strand,” as used herein, refers to the strand of a dsRNA that includes a region that is substantially complementary to a region of the antisense strand.

“Introducing into a cell”, when referring to an oligonucleotide, means facilitating uptake or absorption into the cell, as is understood by those skilled in the art. Absorption or uptake of oligonucleotides can occur through unaided diffusive or active cellular processes, or by auxiliary agents or devices. The meaning of this term is not limited to cells in vitro; an oligonucleotide may also be “introduced into a cell,” wherein the cell is part of a living organism. In such instance, introduction into the cell will include the delivery to the organism. For example, for in vivo delivery, oligonucleotides can be injected into a tissue site or administered systemically. In vivo delivery can also be by a beta-glucan delivery system, such as those described in U.S. Pat. Nos. 5,032,401 and 5,607,677, and U.S. Publication No. 2005/0281781. U.S. Pat. Nos. 5,032,401 and 5,607,677, and U.S. Publication No. 2005/0281781 are hereby incorporated by reference in their entirety. In vitro introduction into a cell includes methods known in the art such as electroporation and lipofection.

The terms “silence” and “inhibit the expression of,” “down-regulate the expression of,” “suppress the expression of,” and the like, in as far as they refer to target gene, herein refer to the at least partial suppression of the expression of the target gene, as manifested by a reduction of the amount of target mRNA, which may be isolated from a first cell or group of cells in which the target gene is transcribed, and which has or have been treated such that the expression of the target gene is inhibited, as compared to a second cell or group of cells substantially identical to the first cell or group of cells but which has or have not been so treated (control cells). The degree of inhibition is usually expressed in terms of

$$\frac{( mRNA in control cells ) - ( mRNA in treated cells )}{( mRNA in control cells )} \cdot 100 \%$$

Alternatively, the degree of inhibition may be given in terms of a reduction of a parameter that is functionally linked to the target gene expression, e.g. the amount of protein encoded by the gene which is secreted by a cell, or the number of cells displaying a certain phenotype, e.g apoptosis. In principle, target gene silencing may be determined in any cell expressing the target, either constitutively or by genomic engineering, and by any appropriate assay. However, when a reference is needed in order to determine whether a given oligonucleotide inhibits the expression of the gene by a certain degree and therefore is encompassed by the instant invention.

For example, in certain instances, expression of the gene is suppressed by at least about 20%, 25%, 30%, 35%, 40%, 45%, or 50% by administration of the compositions having single- or double-stranded oligonucleotides when formulated with ApoE. In some embodiments, the target gene is suppressed by at least about 60%, 70%, or 80% by administration of the compositions comprising the oligonucleotides. In some embodiments, the target gene is suppressed by at least about 85%, 90%, or 95% by of the compositions comprising the oligonucleotides.

As used herein in the context of gene expression the terms “treat”, “treatment,” and the like, refer to relief from or alleviation of pathological processes mediated by target gene expression. As used herein, insofar as it relates to any of the other conditions recited below (other than pathological processes mediated by target gene expression), the terms “treat,” “treatment,” and the like mean to relieve or alleviate at least one symptom associated with such condition, or to slow or reverse the progression of such condition, such as the slowing and progression of hepatic carcinoma.

As used herein, the phrases “therapeutically effective amount” and “prophylactically effective amount” refer to an amount that provides a therapeutic benefit in the treatment, prevention, or management of pathological processes mediated by target gene expression or an overt symptom of pathological processes mediated by target gene expression. The specific amount that is therapeutically effective can be readily determined by ordinary medical practitioner, and may vary depending on factors known in the art, such as, e.g. the type of pathological processes mediated by target gene expression, the patient's history and age, the stage of pathological processes mediated by target gene expression, and the administration of other anti-pathological processes mediated by target gene expression agents.

As used herein, a “pharmaceutical composition” comprises a pharmacologically effective amount of a oligonucleotide preassembled with lipoproteins and optionally a pharmaceutically acceptable carrier. As used herein, “pharmacologically effective amount,” “therapeutically effective amount” or simply “effective amount” refers to that amount of an oligonucleotide preassembled with a lipoproteins effective to produce the intended pharmacological, therapeutic or preventive result. For example, if a given clinical treatment is considered effective when there is at least a 25% reduction in a measurable parameter associated with a disease or disorder, a therapeutically effective amount of a drug for the treatment of that disease or disorder is the amount necessary to effect at least a 25% reduction in that parameter.

The term “pharmaceutically acceptable carrier” refers to a carrier for administration of a therapeutic agent. Such carriers include, but are not limited to, saline, buffered saline, dextrose, water, glycerol, ethanol, and combinations thereof. The term specifically excludes cell culture medium. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

### Pharmaceutical Compositions Comprising Formulated Oligonucleotides

In one embodiment, the invention provides pharmaceutical compositions comprising an oligonucleotide, as described herein, and a pharmaceutically acceptable carrier. The pharmaceutical composition comprising the oligonucleotide is useful for treating a disease or disorder associated with the expression or activity of the target gene, such as pathological processes mediated by target gene expression.

The pharmaceutical compositions featured herein are administered in dosages sufficient to inhibit expression of the target gene.

In general, a suitable dose of total oligonucleotide will be in the range of 0.01 to 200.0 milligrams per kilogram body weight of the recipient per day, generally in the range of 0.02 to 50 mg per kilogram body weight per day. For example, the dsRNA can be administered at 0.01 mg/kg, 0.1 mg/kg, 0.05 mg/kg, 0.5 mg/kg, 1 mg/kg, 2 mg/kg, 3 mg/kg, 5 mg/kg, 10 mg/kg, 20 mg/kg, 30 mg/kg, 40 mg/kg, or 50 mg/kg per single dose. The pharmaceutical composition may be administered once daily or may be administered as two, three, or more sub-doses at appropriate intervals throughout the day or even using continuous infusion or delivery through a controlled release formulation. In that case, the oligonucleotide contained in each sub-dose must be correspondingly smaller in order to achieve the total daily dosage. The dosage unit can also be compounded for delivery over several days, e.g., using a conventional sustained release formulation which provides sustained release of the oligonucleotide over a several day period. Sustained release formulations are well known in the art and are particularly useful for delivery of agents at a particular site, such as could be used with the agents described herein. In this embodiment, the dosage unit contains a corresponding multiple of the daily dose.

The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition can include a single treatment or a series of treatments. Estimates of effective dosages and in vivo half-lives for the individual oligonucleotides encompassed by the invention can be made using conventional methodologies or on the basis of in vivo testing using an appropriate animal model, as described elsewhere herein.

Advances in mouse genetics have generated a number of mouse models for the study of various human diseases, such as pathological processes mediated by target gene expression. Such models are used for in vivo testing of oligonucleotide, as well as for determining a therapeutically effective dose.

The pharmaceutical compositions described herein may be administered in a number of ways depending upon whether local or systemic treatment is desired and upon the area to be treated. Administration may be topical (e.g., by a transdermal patch), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; subdermal, e.g., via an implanted device, or intracranial, e.g., by intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Topical formulations include those in which the dsRNA/ApoE composition is in admixture with a topical delivery agent such as lipids, liposomes, fatty acids, fatty acid esters, steroids, chelating agents and surfactants.

Typical lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA).

Compositions for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Typical oral formulations are those in which the dsRNA/ApoE compositions are administered in conjunction with one or more penetration enhancers surfactants and chelators. Typical surfactants include fatty acids and/or esters or salts thereof, bile acids and/or salts thereof. Typical bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate and sodium glycodihydrofusidate. Typical fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g. sodium). Typical combinations of penetration enhancers include, for example, fatty acids/salts in combination with bile acids/salts. For example, in some embodiments, the combination of the sodium salt of lauric acid, capric acid and UDCA will be used. Other penetration enhancers include polyoxyethylene-9-lauryl ether, polyoxyethylene-20-cetyl ether. DsRNAs may be delivered orally, in granular form including sprayed dried particles, or complexed to form micro or nanoparticles. DsRNA complexing agents include poly-amino acids; polyimines; polyacrylates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pollulans, celluloses and starches. Complexing agents include chitosan, N-trimethylchitosan, poly-L-lysine, polyhistidine, polyornithine, polyspermines, protamine, polyvinylpyridine, polythiodiethylaminomethylethylene P(TDAE), polyaminostyrene (e.g. p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG). Oral formulations for dsRNAs and their preparation are described in detail in U.S. application. Ser. No. 08/886,829 (filed Jul. 1, 1997), Ser. No. 09/108,673 (filed Jul. 1, 1998), Ser. No. 09/256,515 (filed Feb. 23, 1999), Ser. No. 09/082,624 (filed May 21, 1998) and Ser. No. 09/315,298 (filed May 20, 1999), each of which is incorporated herein by reference in their entirety.

Additional compositions useful for parenteral, intrathecal, intraventricular or intrahepatic administration may include sterile aqueous solutions which may also contain buffers, diluents and other suitable additives such as, but not limited to, penetration enhancers, carrier compounds and other pharmaceutically acceptable carriers or excipients.

Suitable pharmaceutical compositions include, but are not limited to, solutions, emulsions, and liposome-containing formulations. These compositions may be generated from a variety of components that include, but are not limited to, preformed liquids, self-emulsifying solids and self-emulsifying semisolids. Suitable formulations will target, for example, one or more of the lung, muscle, heart, or liver.

Pharmaceutical formulations, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

Compositions containing an oligonucleotide associated with ApoE may be formulated into any of many possible dosage forms such as, but not limited to, tablets, capsules, gel capsules, liquid syrups, soft gels, suppositories, and enemas. The compositions may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

Emulsions

The compositions featured herein may be prepared and formulated as emulsions. Emulsions are typically heterogenous systems of one liquid dispersed in another in the form of droplets (Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising two immiscible liquid phases intimately mixed and dispersed with each other. In general, emulsions may be of either the water-in-oil (w/o) or the oil-in-water (o/w) variety. When an aqueous phase is finely divided into and dispersed as minute droplets into a bulk oily phase, the resulting composition is called a water-in-oil (w/o) emulsion. Alternatively, when an oily phase is finely divided into and dispersed as minute droplets into a bulk aqueous phase, the resulting composition is called an oil-in-water (o/w) emulsion. Emulsions may contain additional components in addition to the dispersed phases, and the active drug which may be present as a solution in either the aqueous phase, oily phase or itself as a separate phase. Pharmaceutical excipients such as emulsifiers, stabilizers, dyes, and anti-oxidants may also be present in emulsions as needed. Pharmaceutical emulsions may also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion Likewise a system of oil droplets enclosed in globules of water stabilized in an oily continuous phase provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion may be a semisolid or a solid, as is the case of emulsion-style ointment bases and creams. Other means of stabilizing emulsions entail the use of emulsifiers that may be incorporated into either phase of the emulsion. Emulsifiers may broadly be classified into four categories: synthetic surfactants, naturally occurring emulsifiers, absorption bases, and finely dispersed solids (Idson, in Pharmaceutical Dosage Forms, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants may be classified into different classes based on the nature of the hydrophilic group: nonionic, anionic, cationic and amphoteric (Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that may readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used may be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The application of emulsion formulations via dermatological, oral and parenteral routes and methods for their manufacture have been reviewed in the literature (Idson, in Pharmaceutical Dosage Forms, Lieberman, Rieger and Banker (Eds.), 1988, Marcel Dekker, Inc., New York, N.Y., volume 1, p. 199). Emulsion formulations for oral delivery have been very widely used because of ease of formulation, as well as efficacy from an absorption and bioavailability standpoint (Rosoff, in Pharmaceutical Dosage Forms, Lieberman, Rieger and Banker (Eds.), 1988, Marcel Dekker, Inc., New York, N.Y., volume 1, p. 245; Idson, in Pharmaceutical Dosage Forms, Lieberman, Rieger and Banker (Eds.), 1988, Marcel Dekker, Inc., New York, N.Y., volume 1, p. 199). Mineral-oil base laxatives, oil-soluble vitamins and high fat nutritive preparations are among the materials that have commonly been administered orally as o/w emulsions.

In one embodiment, the compositions are formulated as microemulsions. A microemulsion may be defined as a system of water, oil and amphiphile which is a single optically isotropic and thermodynamically stable liquid solution (Rosoff, in Pharmaceutical Dosage Forms, Lieberman, Rieger and Banker (Eds.), 1988, Marcel Dekker, Inc., New York, N.Y., volume 1, p. 245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in Remington's Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (Rosoff, in Pharmaceutical Dosage Forms, Lieberman, Rieger and Banker (Eds.), 1988, Marcel Dekker, Inc., New York, N.Y., volume 1, p. 245; Block, in Pharmaceutical Dosage Forms, Lieberman, Rieger and Banker (Eds.), 1988, Marcel Dekker, Inc., New York, N.Y., volume 1, p. 335). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (M0310), hexaglycerol monooleate (PO310), hexaglycerol pentaoleate (PO500), decaglycerol monocaprate (MCA750), decaglycerol monooleate (MO750), decaglycerol sequioleate (SO750), decaglycerol decaoleate (DA0750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions may, however, be prepared without the use of cosurfactants and alcohol-free self-emulsifying microemulsion systems are known in the art. The aqueous phase may typically be, but is not limited to, water, an aqueous solution of the drug, glycerol, PEG300, PEG400, polyglycerols, propylene glycols, and derivatives of ethylene glycol. The oil phase may include, but is not limited to, materials such as Captex 300, Captex 355, Capmul MCM, fatty acid esters, medium chain (C₈-C₁₂) mono, di, and tri-glycerides, polyoxyethylated glyceryl fatty acid esters, fatty alcohols, polyglycolized glycerides, saturated polyglycolized C₈-C₁₀ glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (Constantinides et al., Pharmaceutical Research, 1994, 11, 1385-1390; Ritschel, Meth. Find. Exp. Clin. Pharmacol., 1993, 13, 205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (Constantinides et al., Pharmaceutical Research, 1994, 11, 1385; Ho et al., J. Pharm. Sci., 1996, 85, 138-143). Often microemulsions may form spontaneously when their components are brought together at ambient temperature. This may be particularly advantageous when formulating thermolabile drugs, peptides or dsRNAs. Microemulsions have also been effective in the transdermal delivery of active components in both cosmetic and pharmaceutical applications. It is expected that microemulsion compositions and formulations will facilitate the increased systemic absorption of dsRNAs and nucleic acids from the gastrointestinal tract, as well as improve the local cellular uptake of dsRNAs and nucleic acids.

Microemulsions may also contain additional components and additives such as sorbitan monostearate (Grill 3), Labrasol, and penetration enhancers to improve the properties of the formulation and to enhance the absorption of the dsRNAs and nucleic acids featured herein. Penetration enhancers used in the microemulsions may be classified as belonging to one of five broad categories—surfactants, fatty acids, bile salts, chelating agents, and non-chelating non-surfactants (Lee et al., Critical Reviews in Therapeutic Drug Carrier Systems, 1991, p. 92). Each of these classes has been discussed above.

Liposomes

As used in the present invention, the term “liposome” means a vesicle composed of amphiphilic lipids arranged in a spherical bilayer or bilayers.

Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are taken up by macrophages in vivo.

In order to cross intact mammalian skin, lipid vesicles must pass through a series of fine pores, each with a diameter less than 50 nm, under the influence of a suitable transdermal gradient. Therefore, it is desirable to use a liposome which is highly deformable and able to pass through such fine pores.

Further advantages of liposomes include; liposomes obtained from natural phospholipids are biocompatible and biodegradable; liposomes can incorporate a wide range of water and lipid soluble drugs; liposomes can protect encapsulated drugs in their internal compartments from metabolism and degradation (Rosoff, in Pharmaceutical Dosage Forms, Lieberman, Rieger and Banker (Eds.), 1988, Marcel Dekker, Inc., New York, N.Y., volume 1, p. 245). Important considerations in the preparation of liposome formulations are the lipid surface charge, vesicle size and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes and as the merging of the liposome and cell progresses, the liposomal contents are emptied into the cell where the active agent may act.

Liposomal formulations have been the focus of extensive investigation as the mode of delivery for many drugs. There is growing evidence that for topical administration, liposomes present several advantages over other formulations. Such advantages include reduced side-effects related to high systemic absorption of the administered drug, increased accumulation of the administered drug at the desired target, and the ability to administer a wide variety of drugs, both hydrophilic and hydrophobic, into the skin.

Several reports have detailed the ability of liposomes to deliver agents including high-molecular weight DNA into the skin. Compounds including analgesics, antibodies, hormones and high-molecular weight DNAs have been administered to the skin. The majority of applications resulted in the targeting of the upper epidermis

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Several studies have assessed the topical delivery of liposomal drug formulations to the skin. Application of liposomes containing interferon to guinea pig skin resulted in a reduction of skin herpes sores while delivery of interferon via other means (e.g. as a solution or as an emulsion) were ineffective (Weiner et al., Journal of Drug Targeting, 1992, 2, 405-410). Further, an additional study tested the efficacy of interferon administered as part of a liposomal formulation to the administration of interferon using an aqueous system, and concluded that the liposomal formulation was superior to aqueous administration (du Plessis et al., Antiviral Research, 1992, 18, 259-265).

Non-ionic liposomal systems can be used in the delivery of drugs to the skin, in particular systems comprising non-ionic surfactant and cholesterol. Non-ionic liposomal formulations comprising Novasome™ I (glyceryl dilaurate/cholesterol/polyoxyethylene-10-stearyl ether) and Novasome™ II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporin-A into different layers of the skin (Hu et al. S. T. P. Pharma. Sci., 1994, 4, 6, 466).

Liposomes of the compositions also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside GM1, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., FEBS Letters, 1987, 223, 42; Wu et al., Cancer Research, 1993, 53, 3765).

Many liposomes comprising lipids derivatized with one or more hydrophilic polymers, and methods of preparation thereof, are known in the art. Sunamoto et al. (Bull. Chem. Soc. Jpn., 1980, 53, 2778) described liposomes comprising a nonionic detergent, 2C1215G, that contains a PEG moiety. Illum et al. (FEBS Lett., 1984, 167, 79) noted that hydrophilic coating of polystyrene particles with polymeric glycols results in significantly enhanced blood half-lives. Synthetic phospholipids modified by the attachment of carboxylic groups of polyalkylene glycols (e.g., PEG) are described by Sears (U.S. Pat. Nos. 4,426,330 and 4,534,899). Klibanov et al. (FEBS Lett., 1990, 268, 235) described experiments demonstrating that liposomes comprising phosphatidylethanolamine (PE) derivatized with PEG or PEG stearate have significant increases in blood circulation half-lives. Blume et al. (Biochimica et Biophysica Acta, 1990, 1029, 91) extended such observations to other PEG-derivatized phospholipids, e.g., DSPE-PEG, formed from the combination of distearoylphosphatidylethanolamine (DSPE) and PEG. Liposomes having covalently bound PEG moieties on their external surface are described in European Patent No. EP 0 445 131 B1 and WO 90/04384 to Fisher. Liposome compositions containing 1-20 mole percent of PE derivatized with PEG, and methods of use thereof, are described by Woodle et al. (U.S. Pat. Nos. 5,013,556 and 5,356,633) and Martin et al. (U.S. Pat. No. 5,213,804 and European Patent No. EP 0 496 813 B1). Liposomes comprising a number of other lipid-polymer conjugates are disclosed in WO 91/05545 and U.S. Pat. No. 5,225,212 (both to Martin et al.) and in WO 94/20073 (Zalipsky et al.) Liposomes comprising PEG-modified ceramide lipids are described in WO 96/10391 (Choi et al). U.S. Pat. No. 5,540,935 (Miyazaki et al.) and U.S. Pat. No. 5,556,948 (Tagawa et al.) describe PEG-containing liposomes that can be further derivatized with functional moieties on their surfaces.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in Pharmaceutical Dosage Forms, Marcel Dekker, Inc., New York, N.Y., 1988, p. 285).

### Penetration Enhancers

In one embodiment, the compositions featured in the invention employ various penetration enhancers to effect the efficient delivery of nucleic acids, particularly dsRNAs, to the skin of animals. Most drugs are present in solution in both ionized and nonionized forms. However, usually only lipid soluble or lipophilic drugs readily cross cell membranes. It has been discovered that even non-lipophilic drugs may cross cell membranes if the membrane to be crossed is treated with a penetration enhancer. In addition to aiding the diffusion of non-lipophilic drugs across cell membranes, penetration enhancers also enhance the permeability of lipophilic drugs.

Carriers

Certain compositions featured herein may also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The coadministration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioate dsRNA in hepatic tissue can be reduced when it is coadministered with polyinosinic acid, dextran sulfate, polycytidic acid or 4-acetamido-4′ isothiocyano-stilbene-2,2′-disulfonic acid (Miyao et al., DsRNA Res. Dev., 1995, 5, 115-121; Takakura et al., DsRNA & Nucl. Acid Drug Dev., 1996, 6, 177-183.

Excipients

In contrast to a carrier compound, a “pharmaceutical carrier” or “excipient” is a pharmaceutically acceptable solvent, suspending agent or any other pharmacologically inert vehicle for delivering one or more nucleic acids to an animal. The excipient may be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc).

Pharmaceutically acceptable organic or inorganic excipient suitable for non-parenteral administration which do not deleteriously react with nucleic acids can also be used to formulate the compositions featured in the invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids may include sterile and non-sterile aqueous solutions, non-aqueous solutions in common solvents such as alcohols, or solutions of the nucleic acids in liquid or solid oil bases. The solutions may also contain buffers, diluents and other suitable additives. Pharmaceutically acceptable organic or inorganic excipients suitable for non-parenteral administration which do not deleteriously react with nucleic acids can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

Other Components

The compositions featured in the invention may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions, such as dyes, flavoring agents, preservatives, antioxidants, opacifiers, thickening agents and stabilizers. However, such materials, when added, should not unduly interfere with the biological activities of the components of the compositions. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Aqueous suspensions may contain substances which increase the viscosity of the suspension including, for example, sodium carboxymethylcellulose, sorbitol and/or dextran. The suspension may also contain stabilizers.

Certain embodiments featured in the invention provide pharmaceutical compositions containing (a) one or more oligonucleotide compounds and (b) one or more other chemotherapeutic agents which function by a non-antisense mechanism. Examples of such chemotherapeutic agents include but are not limited to daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine, 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine, taxol, vincristine, vinblastine, etoposide (VP-16), trimetrexate, irinotecan, topotecan, gemcitabine, teniposide, cisplatin and diethylstilbestrol (DES). See, generally, The Merck Manual of Diagnosis and Therapy, 15th Ed. 1987, pp. 1206-1228, Berkow et al., eds., Rahway, N.J. When used with a dsRNA/ApoE complex, such chemotherapeutic agents may be used individually (e.g., 5-FU and oligonucleotide), sequentially (e.g., 5-FU and oligonucleotide for a period of time followed by MTX and oligonucleotide), or in combination with one or more other such chemotherapeutic agents (e.g., 5-FU, MTX and oligonucleotide, or 5-FU, radiotherapy and oligonucleotide). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in the compositions described herein. See, generally, The Merck Manual of Diagnosis and Therapy, 15th Ed., Berkow et al., eds., 1987, Rahway, N.J., pages 2499-2506 and 46-49, respectively). Other non-antisense chemotherapeutic agents are also within the scope of this invention. Two or more combined compounds may be used together or sequentially.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are typical.

The data obtained from cell culture assays and animal studies can be used in formulation a range of dosage for use in humans. The dosage of the composition generally lies within a range of circulating concentrations that include the ED50 with little or no toxicity. The dosage may vary within this range depending upon the dosage form employed and the route of administration utilized. For any compound used as described herein, the therapeutically effective dose can be estimated initially from cell culture assays. A dose may be formulated in animal models to achieve a circulating plasma concentration range of the compound or, when appropriate, of the polypeptide product of a target sequence (e.g., achieving a decreased concentration of the polypeptide) that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In addition to their administration individually or as a plurality, as discussed above, the dsRNAs featured in the invention can be administered in combination with other known agents effective in treatment of pathological processes mediated by target gene expression. In any event, the administering physician can adjust the amount and timing of oligonucleotide administration on the basis of results observed using standard measures of efficacy known in the art or described herein.

Methods for Treating Diseases Caused by Expression of a Target Gene Using the Dsrnas Complexed with ApoE

The invention relates in particular to compositions comprising at least one of a single or double stranded oligonucleotide, where said oligonucleotide has been conjugated to a lipophile to which the conjugated oligonucleotide has been preassembled with a lipoprotein for the treatment of a disease. The lipoprotein is, for example, an ApoE, e.g., an isoform or mixture of isoforms of ApoE, such as one or more of ApoE2, ApoE3, or ApoE4. In one embodiment, the described compositions can be used in combination with other known treatments to treat conditions or diseases. For example, the described compositions can be used in combination with one or more known therapeutic agents to treat breast, lung, prostate, colorectal, brain, esophageal, bladder, pancreatic, cervical, head and neck, and ovarian cancer; melanoma, lymphoma, glioma, multidrug resistant cancers, and/or HIV, HBV, HCV, CMV, RSV, HSV, poliovirus, influenza, rhinovirus, west nile virus, Ebola virus, foot and mouth virus, papilloma virus, and SARS virus infection, other cancers and other infectious diseases, autoimmunity, inflammation, endocrine disorders, renal disease, pulmonary disease, cardiovascular disease, CNS injury, CNS disease, neurodegenerative disease, birth defects, aging, any other disease or condition related to gene expression.

The invention furthermore relates to the use of dsRNA complexed with an ApoE, e.g., for treating cancer or for preventing tumor metastasis, in combination with other pharmaceuticals and/or other therapeutic methods, e.g., with known pharmaceuticals and/or known therapeutic methods, such as, for example, those which are currently employed for treating cancer and/or for preventing tumor metastasis. Preference is given to a combination with radiation therapy and chemotherapeutic agents, such as cisplatin, cyclophosphamide, 5-fluorouracil, adriamycin, daunorubicin or tamoxifen.

The invention can also be practiced by including with a specific oligonucleotide, in combination with another anti-cancer chemotherapeutic agent, such as any conventional chemotherapeutic agent. The combination of a specific binding agent with such other agents can potentiate the chemotherapeutic protocol. Numerous chemotherapeutic protocols will present themselves in the mind of the skilled practitioner as being capable of incorporation into the methods featured in the invention. Any chemotherapeutic agent can be used, including alkylating agents, antimetabolites, hormones and antagonists, radioisotopes, as well as natural products. For example, a dsRNA complexed with ApoE can be administered with antibiotics such as doxorubicin and other anthracycline analogs, nitrogen mustards such as cyclophosphamide, pyrimidine analogs such as 5-fluorouracil, cisplatin, hydroxyurea, taxol and its natural and synthetic derivatives, and the like. As another example, in the case of mixed tumors, such as adenocarcinoma of the breast, where the tumors include gonadotropin-dependent and gonadotropin-independent cells, the compound can be administered in conjunction with leuprolide or goserelin (synthetic peptide analogs of LH-RH). Other antineoplastic protocols include the use of a tetracycline compound with another treatment modality, e.g., surgery, radiation, etc., also referred to herein as “adjunct antineoplastic modalities.” Thus, the methods featured in the invention can be employed with such conventional regimens with the benefit of reducing side effects and enhancing efficacy.

In some embodiments, a composition containing a particle that comprises an oligonucleotide in combination with an ApoE, e.g., a recombinant ApoE, is suitable for treating a lipid or metabolic disorder, such as hypercholesterolemia, dyslipidemia, diabetes, diabetes type I, diabetes type II, coronary artery disease, atherosclerosis, myocardial infarction, coronary artery bypass graft, percutaneous transluminal angioplasties, coronary stenosis, cerebrovascular disease transient ischemic attack, ischemic stroke, carotid endarterectomies, peripheral arterial disease, and other disorders associated with cholesterol metabolism.

In some embodiments, an oligonucleotide complexed with an ApoE can be administered in combination with a second agent to treat the lipid or metablic disorder. For example, the composition comprising an oligonucleotide complexed with an ApoE can be administered in combination with an HMG-CoA reductase inhibitor (e.g., a statin, such as atrovastatin, lovastatin, pravastatin or simvastatin), a fibrate, a bile acid sequestrant, niacin, an antiplatelet agent, an angiotensin converting enzyme inhibitor, an angiotensin II receptor antagonist (e.g., losartan potassium, such as Merck & Co.'s Cozaar®), an acylCoA cholesterol acetyltransferase (ACAT) inhibitor, a cholesterol absorption inhibitor, a cholesterol ester transfer protein (CETP) inhibitor, a microsomal triglyceride transfer protein (MTTP) inhibitor, a cholesterol modulator, a bile acid modulator, a peroxisome proliferation activated receptor (PPAR) agonist, a gene-based therapy, a composite vascular protectant (e.g., AGI-1067, from Atherogenics), a glycoprotein IIb/IIIa inhibitor, aspirin or an aspirin-like compound, an IBAT inhibitor (e.g., S-8921, from Shionogi), a squalene synthase inhibitor, or a monocyte chemoattractant protein (MCP)-I inhibitor.

### Methods for Inhibiting Target Gene Expression

In yet another aspect, the invention provides a method for inhibiting the expression of the target gene in a mammal. The method includes administering a composition featured in the invention to the mammal such that expression of the target gene is silenced.

In one embodiment, a method for inhibiting target gene expression includes administering a composition containing a nucleotide sequence that is complementary to at least a part of an RNA transcript of the target gene and the other having a nucleotide sequence that is complementary to at least a part of an RNA transcript of the gene of the mammal to be treated. When the organism to be treated is a mammal such as a human, the composition may be administered by any means known in the art including, but not limited to oral or parenteral routes, including intravenous, intramuscular, subcutaneous, transdermal, airway (aerosol), nasal, rectal, and topical (including buccal and sublingual) administration. In certain embodiments, the compositions are administered by intravenous infusion or injection.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

Related information is presented in Wolfrum, et al., Nature Biotechnology 25, 1149-1157 (2007), which is herein incorporated by reference in its entirety.

### Example 1

### Oligonucleotide Synthesis

### Source of Reagents

Where the source of a reagent is not specifically given herein, such reagent may be obtained from any supplier of reagents for molecular biology at a quality/purity standard for application in molecular biology.

siRNA (dsRNA) Synthesis

Single-stranded RNAs were produced by solid phase synthesis on a scale of 1 μmole using an Expedite 8909 synthesizer (Applied Biosystems, Applera Deutschland GmbH, Darmstadt, Germany) and controlled pore glass (CPG, 500 Å, Proligo Biochemie GmbH, Hamburg, Germany) as solid support. RNA and RNA containing 2′-O-methyl nucleotides were generated by solid phase synthesis employing the corresponding phosphoramidites and 2′-O-methyl phosphoramidites, respectively (Proligo Biochemie GmbH, Hamburg, Germany). These building blocks were incorporated at selected sites within the sequence of the oligoribonucleotide chain using standard nucleoside phosphoramidite chemistry such as described in Current protocols in nucleic acid chemistry, Beaucage, S. L. et al. (Edrs.), John Wiley & Sons, Inc., New York, N.Y., USA. Phosphorothioate linkages were introduced by replacement of the iodine oxidizer solution with a solution of the Beaucage reagent (Chruachem Ltd, Glasgow, UK) in acetonitrile (1%). Further ancillary reagents were obtained from Mallinckrodt Baker (Griesheim, Germany).

Deprotection and purification of the crude oligoribonucleotides by anion exchange HPLC were carried out according to established procedures. Yields and concentrations were determined by UV absorption of a solution of the respective RNA at a wavelength of 260 nm using a spectral photometer (DU 640B, Beckman Coulter GmbH, UnterschleiBheim, Germany). Double stranded RNA was generated by mixing an equimolar solution of complementary strands in annealing buffer (20 mM sodium phosphate, pH 6.8; 100 mM sodium chloride), heated in a water bath at 85-90° C. for 3 minutes and cooled to room temperature over a period of 3-4 hours. The annealed RNA solution was stored at −20° C. until use.

For the synthesis of 3′-cholesterol-conjugated siRNAs (herein referred to as -Chol-3′), an appropriately modified solid support was used for RNA synthesis. The modified solid support was prepared as follows:

### Diethyl-2-azabutane-1,4-dicarboxylate AA

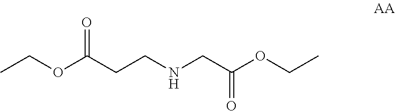

A 4.7 M aqueous solution of sodium hydroxide (50 mL) was added into a stirred, ice-cooled solution of ethyl glycinate hydrochloride (32.19 g, 0.23 mole) in water (50 mL). Then, ethyl acrylate (23.1 g, 0.23 mole) was added and the mixture was stirred at room temperature until completion of the reaction was ascertained by TLC. After 19 h the solution was partitioned with dichloromethane (3×100 mL). The organic layer was dried with anhydrous sodium sulfate, filtered and evaporated. The residue was distilled to afford AA (28.8 g, 61%).

### 3-{Ethoxycarbonylmethyl-[6-(9H-fluoren-9-ylmethoxycarbonyl-amino)-hexanoyl]-amino}-propionic acid ethyl ester AB

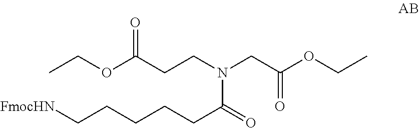

Fmoc-6-amino-hexanoic acid (9.12 g, 25.83 mmol) was dissolved in dichloromethane (50 mL) and cooled with ice. Diisopropylcarbodiimde (3.25 g, 3.99 mL, 25.83 mmol) was added to the solution at 0° C. It was then followed by the addition of Diethyl-azabutane-1,4-dicarboxylate (5 g, 24.6 mmol) and dimethylamino pyridine (0.305 g, 2.5 mmol). The solution was brought to room temperature and stirred further for 6 h. Completion of the reaction was ascertained by TLC. The reaction mixture was concentrated under vacuum and ethyl acetate was added to precipitate diisopropyl urea. The suspension was filtered. The filtrate was washed with 5% aqueous hydrochloric acid, 5% sodium bicarbonate and water. The combined organic layer was dried over sodium sulfate and concentrated to give the crude product which was purified by column chromatography (50% EtOAC/Hexanes) to yield 11.87 g (88%) of AB.

### 3-[(6-Amino-hexanoyl)-ethoxycarbonylmethyl-amino]-propionic acid ethyl ester AC

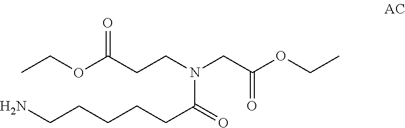

3-{Ethoxycarbonylmethyl-[6-(9H-fluoren-9-ylmethoxycarbonylamino)-hexanoyl]-amino}-propionic acid ethyl ester AB (11.5 g, 21.3 mmol) was dissolved in 20% piperidine in dimethylformamide at 0° C. The solution was continued stiffing for 1 h. The reaction mixture was concentrated under vacuum, water was added to the residue, and the product was extracted with ethyl acetate. The crude product was purified by conversion into its hydrochloride salt.

### 3-({6-[17-(1,5-Dimethyl-hexyl)-10,13-dimethyl-2,3,4,7,8,9,10,11,12,13,14,15,16,17-tetradecahydro-1H-cyclopenta[α]phenanthren-3-yloxycarbonylamino]-hexanoyl}ethoxycarbonylmethyl-amino)-propionic acid ethyl ester AD

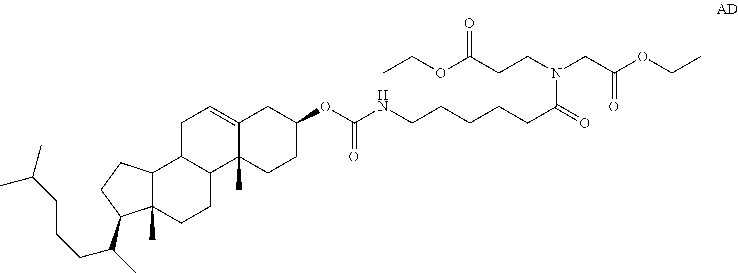

The hydrochloride salt of 3-[(6-Amino-hexanoyl)-ethoxycarbonylmethyl-amino]-propionic acid ethyl ester AC (4.7 g, 14.8 mmol) was taken up in dichloromethane. The suspension was cooled to 0° C. on ice. To the suspension diisopropylethylamine (3.87 g, 5.2 mL, 30 mmol) was added. To the resulting solution cholesteryl chloroformate (6.675 g, 14.8 mmol) was added. The reaction mixture was stirred overnight. The reaction mixture was diluted with dichloromethane and washed with 10% hydrochloric acid. The product was purified by flash chromatography (10.3 g, 92%).

### 1-{6-[17-(1,5-Dimethyl-hexyl)-10,13-dimethyl-2,3,4,7,8,9,10,11,12,13,14,15,16,17-tetradecahydro-1H-cyclopenta[a]phenanthren-3-yloxycarbonylamino]-hexanoyl}-4-oxo-pyrrolidine-3-carboxylic acid ethyl ester AE

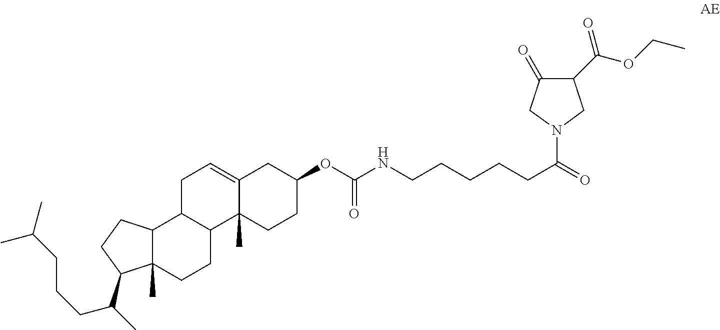

Potassium t-butoxide (1.1 g, 9.8 mmol) was slurried in 30 mL of dry toluene. The mixture was cooled to 0° C. on ice and 5 g (6.6 mmol) of diester AD was added slowly with stiffing within 20 mins. The temperature was kept below 5° C. during the addition. The stirring was continued for 30 mins at 0° C. and 1 mL of glacial acetic acid was added, immediately followed by 4 g of NaH₂PO₄.H₂O in 40 mL of water The resultant mixture was extracted twice with 100 mL of dichloromethane each and the combined organic extracts were washed twice with 10 mL of phosphate buffer each, dried, and evaporated to dryness. The residue was dissolved in 60 mL of toluene, cooled to 0° C. and extracted with three 50 mL portions of cold pH 9.5 carbonate buffer. The aqueous extracts were adjusted to pH 3 with phosphoric acid, and extracted with five 40 mL portions of chloroform which were combined, dried and evaporated to dryness. The residue was purified by column chromatography using 25% ethylacetate/hexane to afford 1.9 g of b-ketoester (39%).

### [6-(3-Hydroxy-4-hydroxymethyl-pyrrolidin-1-yl)-6-oxo-hexyl]-carbamic acid 17-(1,5-dimethyl-hexyl)-10,13-dimethyl-2,3,4,7,8,9,10,11,12,13,14,15,16,17-tetradecahydro-1H-cyclopenta[a]phenanthren-3-yl ester AF

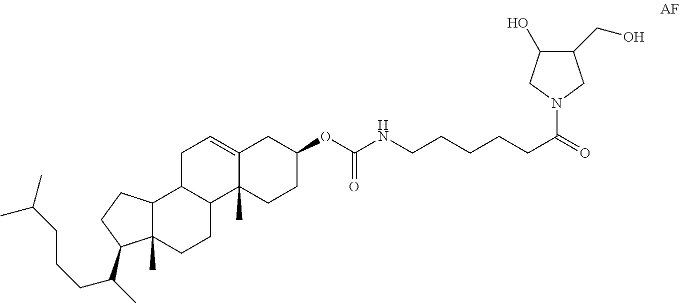

Methanol (2 mL) was added dropwise over a period of 1 h to a refluxing mixture of b-ketoester AE (1.5 g, 2.2 mmol) and sodium borohydride (0.226 g, 6 mmol) in tetrahydrofuran (10 mL). Stirring was continued at reflux temperature for 1 h. After cooling to room temperature, 1 N HCl (12.5 mL) was added, the mixture was extracted with ethylacetate (3×40 mL). The combined ethylacetate layer was dried over anhydrous sodium sulfate and concentrated under vacuum to yield the product which was purified by column chromatography (10% MeOH/CHCl₃) (89%).

### (6-{3-[Bis-(4-methoxy-phenyl)-phenyl-methoxymethyl]-4-hydroxy-pyrrolidin-1-yl}-6-oxo-hexyl)-carbamic acid 17-(1,5-dimethyl-hexyl)-10,13-dimethyl-2,3,4,7,8,9,10,11,12,13,14,15,16,17-tetradecahydro-1H-cyclopenta[α]phenanthren-3-yl ester AG

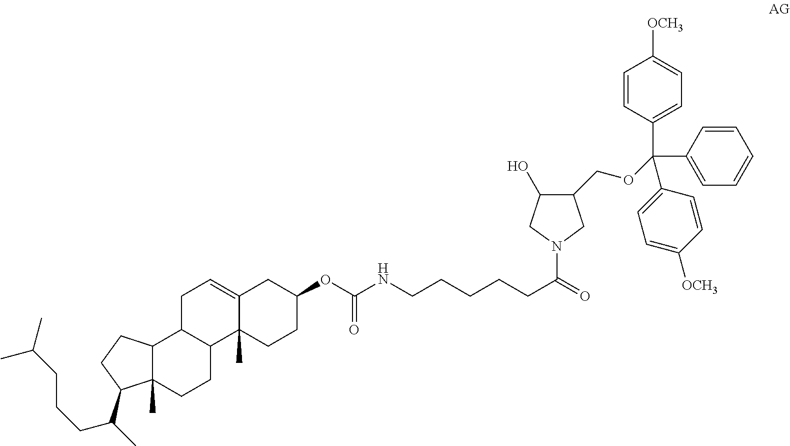

Diol AF (1.25 gm 1.994 mmol) was dried by evaporating with pyridine (2×5 mL) in vacuo. Anhydrous pyridine (10 mL) and 4,4′-dimethoxytritylchloride (0.724 g, 2.13 mmol) were added with stirring. The reaction was carried out at room temperature overnight. The reaction was quenched by the addition of methanol. The reaction mixture was concentrated under vacuum and to the residue dichloromethane (50 mL) was added. The organic layer was washed with 1M aqueous sodium bicarbonate. The organic layer was dried over anhydrous sodium sulfate, filtered and concentrated. The residual pyridine was removed by evaporating with toluene. The crude product was purified by column chromatography (2% MeOH/Chloroform, R_{f}=0.5 in 5% MeOH/CHCl₃) (1.75 g, 95%).

### Succinic acid mono-(4-[bis-(4-methoxy-phenyl)-phenyl-methoxymethyl]-1-{6-[17-(1,5-dimethyl-hexyl)-10,13-dimethyl 2,3,4,7,8,9,10,11,12,13,14,15,16,17-tetradecahydro-1H cyclopenta[a]phenanthren-3-yloxycarbonylamino]-hexanoyl}-pyrrolidin-3-yl) ester AH

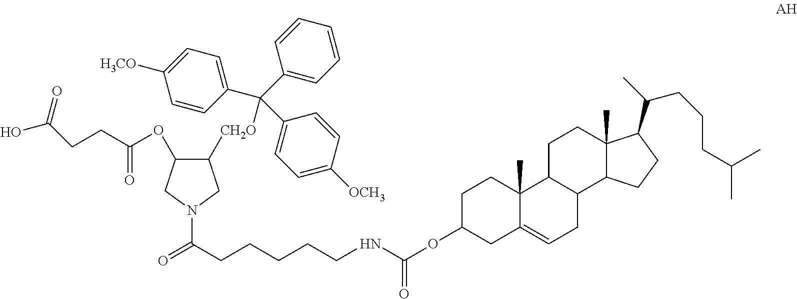

Compound AG (1.0 g, 1.05 mmol) was mixed with succinic anhydride (0.150 g, 1.5 mmol) and DMAP (0.073 g, 0.6 mmol) and dried in a vacuum at 40° C. overnight. The mixture was dissolved in anhydrous dichloroethane (3 mL), triethylamine (0.318 g, 0.440 mL, 3.15 mmol) was added and the solution was stirred at room temperature under argon atmosphere for 16 h. It was then diluted with dichloromethane (40 mL) and washed with ice cold aqueous citric acid (5 wt %, 30 mL) and water (2×20 mL). The organic phase was dried over anhydrous sodium sulfate and concentrated to dryness. The residue was used as such for the next step.

### Cholesterol derivatised CPG AI

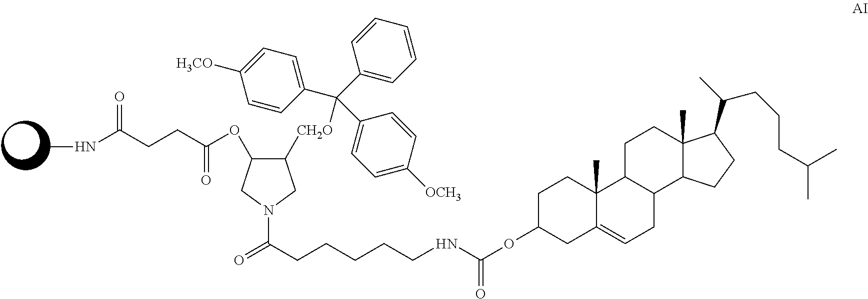

Succinate AH (0.254 g, 0.242 mmol) was dissolved in a mixture of dichloromethane/acetonitrile (3:2, 3 mL). To that solution DMAP (0.0296 g, 0.242 mmol) in acetonitrile (1.25 mL), 2,2′-Dithio-bis(5-nitropyridine) (0.075 g, 0.242 mmol) in acetonitrile/dichloroethane (3:1, 1.25 mL) were added successively. To the resulting solution triphenylphosphine (0.064 g, 0.242 mmol) in acetonitrile (0.6 ml) was added. The reaction mixture turned bright orange in color. The solution was agitated briefly using a wrist-action shaker (5 mins). Long chain alkyl amine-CPG (LCAA-CPG) (1.5 g, 61 mM) was added. The suspension was agitated for 2 h. The CPG was filtered through a sintered funnel and washed with acetonitrile, dichloromethane and ether successively. Unreacted amino groups were masked using acetic anhydride/pyridine. The achieved loading of the CPG was measured by taking UV measurement (37 mM/g).

The synthesis of siRNAs bearing a 5′-12-dodecanoic acid bisdecylamide group (herein referred to as “5′-C32-”) or a 5′-cholesteryl derivative group (herein referred to as “5′-Chol-”) was performed as described in WO 2004/065601, except that, for the cholesteryl derivative, the oxidation step was performed using the Beaucage reagent in order to introduce a phosphorothioate linkage at the 5′-end of the nucleic acid oligomer.

Nucleic acid sequences are represented using standard nomenclature, and specifically the abbreviations of Table 1.

TABLE 1
Abbreviations of nucleotide monomers used in nucleic
acid sequence representation. It will be understood that
these monomers, when present in an oligonucleotide, are
mutually linked by 5′-3′-phosphodiester bonds.

| Abbreviation | Nucleotide(s) |
|---|---|
| A | adenosine |
| C | cytidine |
| G | guanosine |
| T | thymidine |
| U | uridine |
| N | any nucleotide (G, A, C, U T) |
| s or subscript ‘s’ | Phosphorothioate linkage |
| sT | 2′-deoxy-thymidine-5′phosphate-phosphorothioate |
| L | the lipophile |
| L10 | N-(cholesterylcarboxamidocaproyl)-4-hydroxyprolinol |
| | (Hyp-C6-Chol) |
| Chol | Cholesterol |
| P | phosphate group |
| fC | 2′-deoxy-2′-fluoro cytidine |
| fU | 2′-deoxy-2′-fluoro uridine |
| u, c, g, a | lower case letters, 2′-O-methyl sugar modification |

### Example 2

### Synthesis of Recombinant ApoE3

The ApoE3 gene was engineered to optimize codon usage, and ApoE3 was expressed as a fusion protein in E. coli. The fusion protein, thioredoxin-Stag-8HIS-AcTEV-ApoE, was cloned into pET32a(+) and transformed into E. coli expression strain BL21 (DE3). Cells were grown until at OD600=0.8 and protein expression was induced by addition of IPTG (0.5 mM). Cells were grown for additional 2 hours and harvested by centrifugation. Cells were lysed by one round of freeze-thaw followed by sonication in 6 M guanidine, 60 mM TRIS, pH 8.0, 1 mM β-mercaptoethanol. The lysis mixture was cleared by centrifugation and imidazole was added to the supernatant (5 mM). This mixture was loaded onto a Ni-Sepharose6 FF column (GE Healthcare) and the column was extensively washed with buffer (50 mM Tris, pH 8.0, 5 M guanidine, 5 mM imidazole, 1 mM 13-mercaptoethanol, 1% Triton-X114). The column was then washed with buffer A (40 mM TRIS, pH 8.0, 4 M guanidine, 1 mM (3-mercaptoethanol, 5 mM imidazole). Fusion protein was eluted with buffer B (buffer A+0.4 M imidazole) using a linear concentration gradient. After dialyzing against 100 mM ammonium bicarbonate, the thioredoxin-Stag-8HIS-AcTEV moiety was removed by cleavage with AcTEV protease (Invitrogen). ApoE was isolated by passing the mixture over the Ni-Sepharose6 FF column and further purified by heparin-Sepharose affinity chromatography. The yield was approximately 30 mg/L and the endotoxin levels were 1-5 EU/mg.

ApoE3 was also produced in HEK293 cells. Specifically, one liter culture of HEK293 cells was transiently transfected with ApoE-pcDNA3.1(+) containing human ApoE cDNA (Origene Technologies) using 293 Fectin (Invitrogen). Every 48 h after transfection, 30% of the media was replaced with fresh media and the culture was continued for 170 hours. Media was pooled, concentrated by tangential flow filtration, and loaded onto a heparin-Sepharose column. The column was washed with 10 mM sodium phosphate buffer pH 7.6 containing 1 mM DTT (buffer A) and ApoE was eluted with buffer A containing 1M NaCl. The yield was approximately 30 mg/L and the endotoxin level was approximately 1 EU/mg.

Table 2 compares the expression of ApoE3 in HEK293 cells and E. coli.

TABLE 2
Expression of ApoE3 in HEK293 cells and E. coli

| | HEK293 | E. coli |
|---|---|---|
| Expression | Secreted into medium | Expressed as thioredoxin-ApoE |
| | Native sequence | fusion Extra Gly at N-terminus |
| Glycosylation | Yes | No |
| Purification | Heparin affinity | NTA affinity in 6M guanidine |
| | | Thioredoxin removed by |
| | | proteolysis |
| Endotoxin | 10 eu/mg | Removed during purification, |
| | | 10 eu/mg |
| Yield (after | 30 mg/L | 30 mg/L |
| purification) | | |

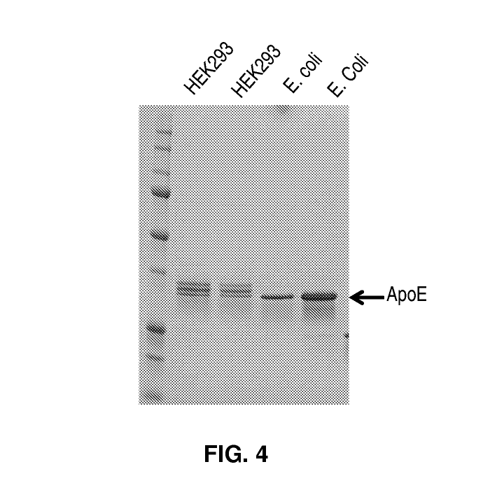

FIG. 4 is an image of SDS-PAGE showing the expression of ApoE in E. coli and HEK293 cells. Amino acid sequence of ApoE from HEK293 cells showed the N-terminal squence of all three bands are the same, indicating the top two bands are glycosylated forms.

### Example 3

### Reconstitution of ApoE-rHDL with POPC

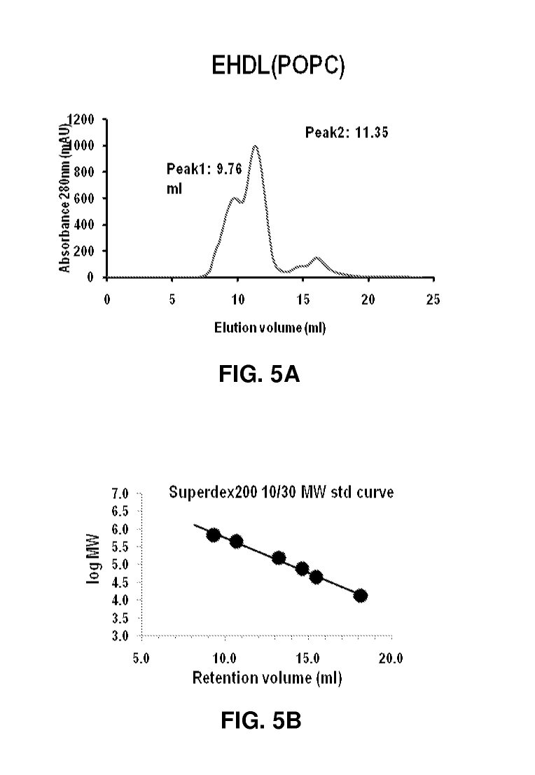

A reconstituted ApoE-HDL complex (rEHDL) was prepared by the sodium cholate dialysis method (Jonas et al., JBC 264:4818-4824, 1989) with ApoE and POPC in molar ratio of 160:1 (POPC:ApoE). The resulting complexes were analyzed as shown in Table 3. The characteristics of the elution profile are illustrated by graphs in FIGS. 5A and 5B.

TABLE 3
Analysis of ApoE-rHDL constituted with POPC

| Analysis | Result |
|---|---|
| POPC and | Peak 1: 200:1, POPC:ApoE |
| ApoE analysis | Peak 2: 170:1, POPC:ApoE |
| Gel filtration | Peak 1: Retention vol = 9.8 ml; |
| (Superdex200 | MW ~630 kD |
| 10/30) | Peak 2: Retention vol = 11 ml |
| | MW ~308 kD |
| Dynamic light | Peak 1: Diameter = 16 nm |
| scattering | Peak 2: Diameter = 12 nm |

Reconstitution of ApoE-rHDL with DMPC (Dimyristoylphosphatidylcholine)

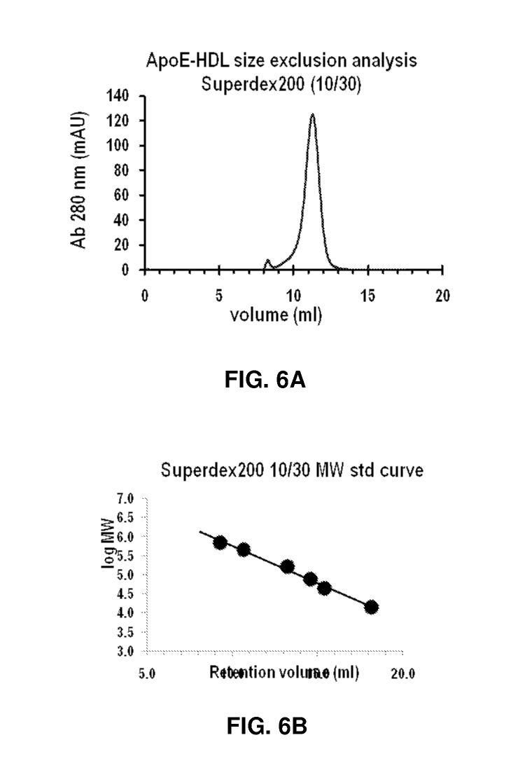

EHDL was prepared by Na cholate dialysis method (Jonas et al., JBC 264:4818-4824, 1989) with ApoE and DMPC in molar ratio of 190:1 (DMPC:ApoE). The resulting complexes were analyzed as shown in Table 4. The characteristics of the elution profile are shown in FIGS. 6A and 6B.

TABLE 4
Analysis of ApoE-rHDL constituted with DMPC

| Analysis | Result |
|---|---|
| DMPC and | 1) 192:1, DMPC:ApoE |
| ApoE analysis | |
| Gel filtration | Retention volume = 11.28 ml; |
| (Superdex200 | MW ~318 kD (if 2 ApoE per |
| 10/30) | particle, calculated MW ~329 kD) |
| Dynamic light | Diameter = 10.8 nm |
| scattering | |

ApoE-rHDL/AD5167 Particle

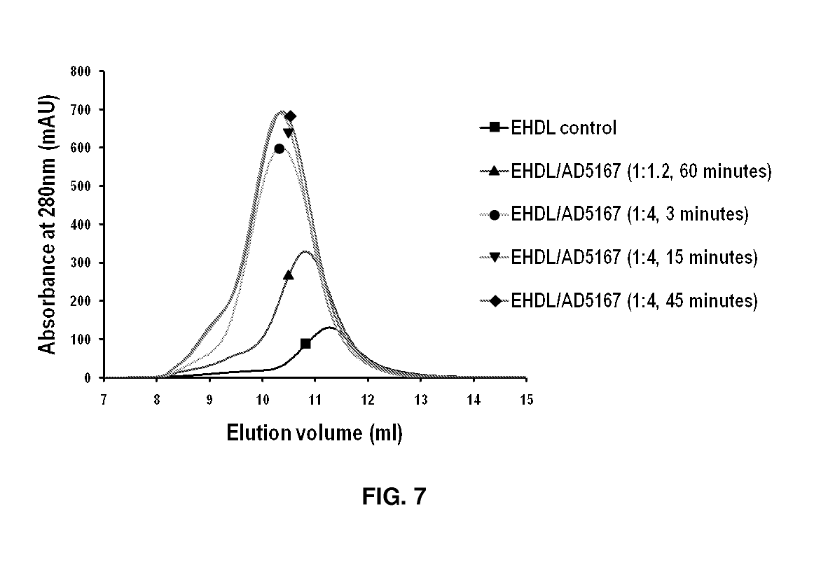

EHDL was mixed with the dsRNA AD5167 (see Table 5) at different molar ratios (1:1.2 or 1:4 (EHDL/AD5167)) and incubated for 3, 15, 45 or 60 minutes. The resulting ApoE-rHDL/AD5167 particles were characterized by size exclusion analysis (FIG. 7). Cholesterol-siRNA was loaded onto EHDL at ˜1:1 stoichiometry for in vivo studies.

TABLE 5
AD5167 and AD5544 Duplex

| | | | |
|---|---|---|---|
| AD5167 | (S) | 5'-GUCAUCACACUGAAUACCAAUsL10-3' | SEQ ID NO: 14 |
| | (AS) | 5'-AUUGGUAUUCAGUGUGAUGACsAsC-3' | SEQ ID NO: 15 |
| AD5544 | (S) | 5'-GGAAUCuuAuAuuuGAUCcAAsL10-3' | SEQ ID NO: 16 |
| | (AS) | 5'-uuGGAUcAAAuAuAAGAuUCcscsU-3' | SEQ ID NO: 17 |

### Example 4

### ApoE3-Based Reconstituted HDL Inhibited ApoB Gene Expression In Vivo

ApoE-based rHDL (called rEHDL) was prepared from recombinant ApoE3 from E. coli and DMPC by the cholate dialysis method (Matz and Jones, Jour. Biol. Chem. 257:4535, 1982). The particle size was 12 nm by Malvern NanoZS and 300 kDa by size-exclusion chromatography. The rEHDL:siRNA binding ratio was 1:1.25.

C57BL6, ApoE−/− and LDLR−/− mice (6-8 weeks old) were administered AD5167 (30 mg/kg) by intravenous administration in a single bolus dose. Four test mice were administered AD5167 and two control mice were administered PBS. Mice were fasted overnight before being sacrificed at 48 h post-injection. Plasma apoB protein levels were determined by Western blot analysis. Liver and jejunum apoB mRNA levels were determined by bDNA assay and quantitative PCR (qPCR). Serum cholesterol levels were determined by IDEXX VetTest.

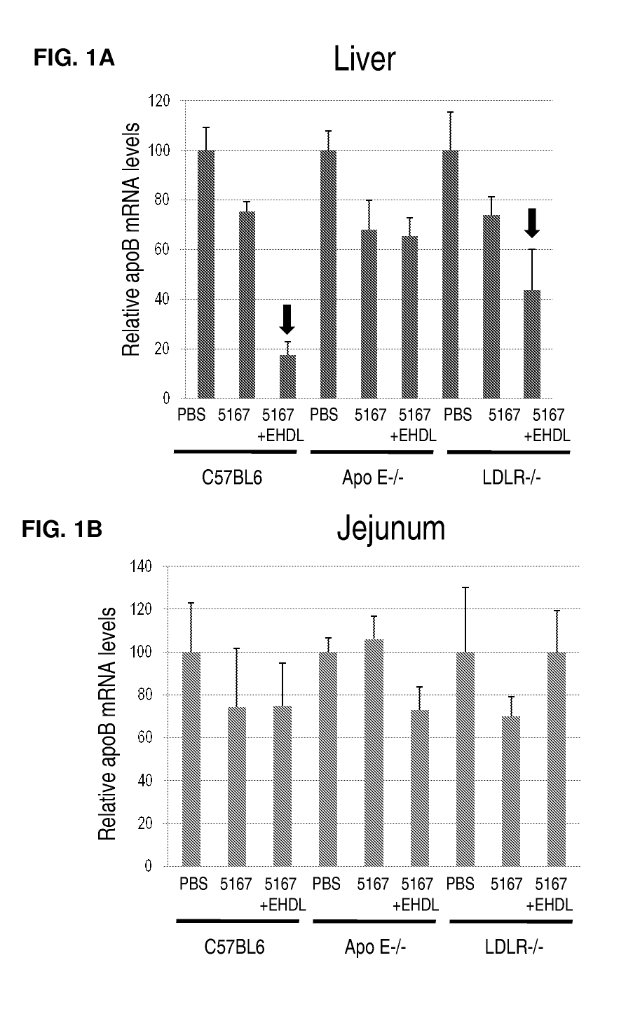

As shown in FIG. 1A, AD5167 complexed with rEHDL decreased levels of ApoB mRNA by nearly 85% in liver of C57BL6 mice. No facilitation of ApoB mRNA knockdown was observed in ApoE−/− mice, suggesting that abnormal lipoprotein metabolism might affect ApoE-HDL siRNA delivery. FIGS. 1A and 1B show the results of bDNA assays, and quantitative PCR (qPCR) assays yielded similar results. There was little or no knockdown of jejunum apoB mRNA in C57BL6, ApoE knockout, and LDL receptor (LDLR) knockout mice.

Reduction of liver ApoB mRNA levels was reflected by reduced serum ApoB protein and cholesterol levels.

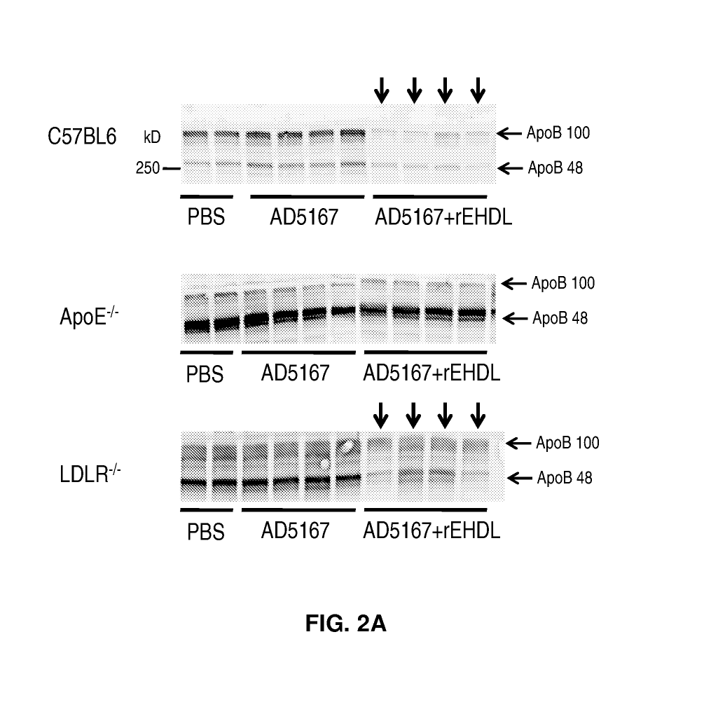

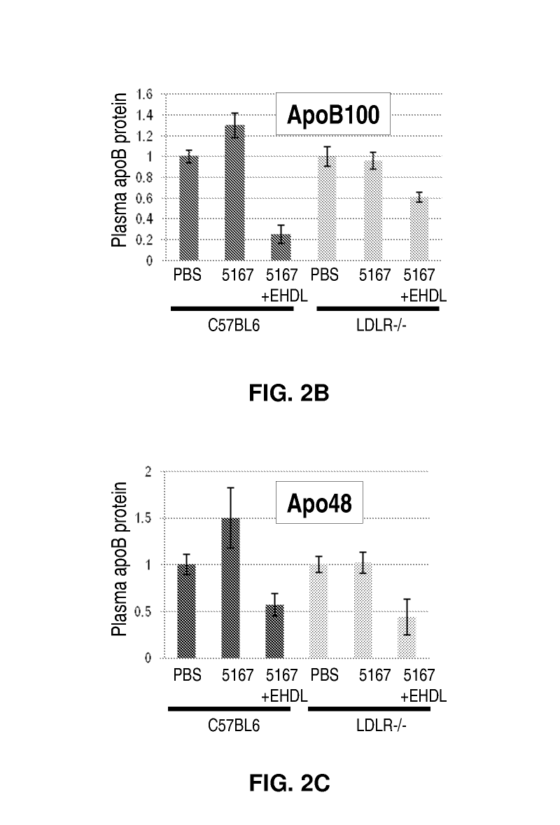

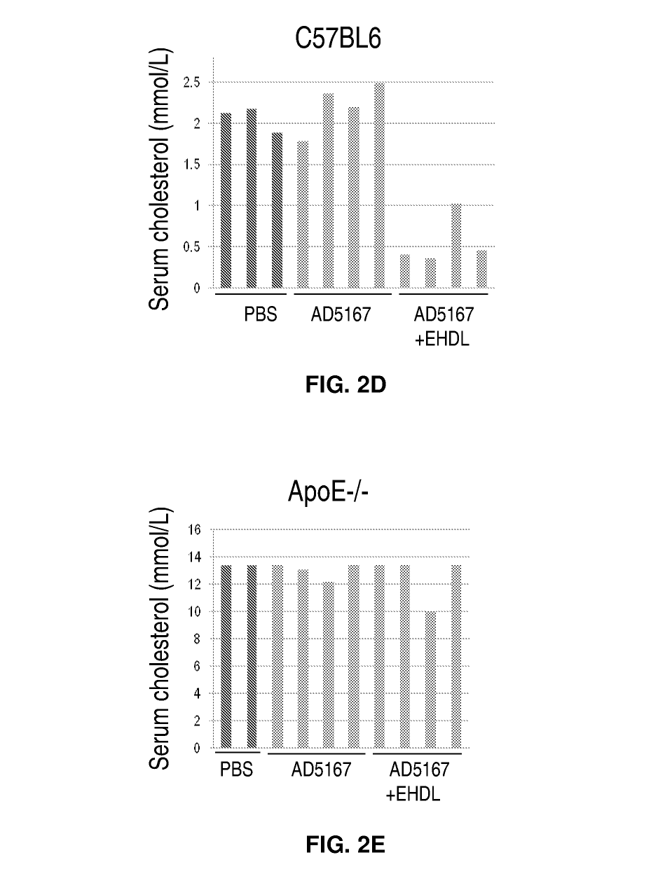

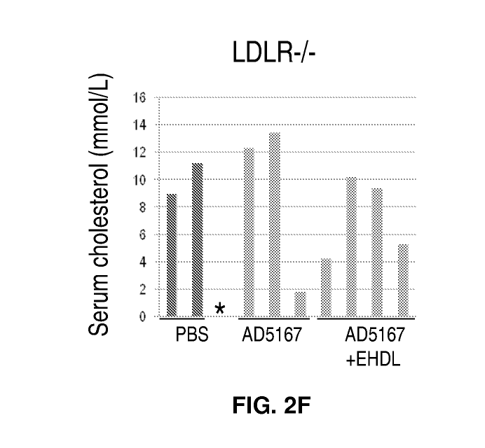

As shown in FIGS. 2A-2C, plasma ApoB levels were also reduced in C57BL6 and LDLR knockout mice. The decrease in LDLR−/− mice was about 60%, which was lower than that observed for wildtype mice. This is likely due to some dependence on the LDL receptor. The decrease in plasma ApoB levels was not observed in ApoE knockout mice.

As shown in FIGS. 2D-2F, serum cholestrol levels were also reduced in C57BL6 and LDLR knockout mice. The decrease in plasma serum cholesterol levels was not observed in ApoE knockout mice. The mouse normal serum cholesterol range is from 0.93 to 2.48 mmol/L.

### Example 5

### Dose Response Study of the Inhibition of ApoB Gene Expression by ApoE3-Based Reconstituted HDL In Vivo

ApoE-based rHDL (rEHDL) was prepared from recombinant ApoE3 from HEK293 cells.

C57BL6 male mice (7-8 weeks old, n=4) were administered AD5167 complexed with rEHDL at a dose of 1, 3, 10 and 30 mg/kg by intravenous administration in a single bolus dose. Mice were fasted overnight before being sacrificed at 48 h post-injection. Plasma apoB protein levels were determined by Western blot analysis. Liver and jejunum apoB mRNA levels were determined by bDNA assay and quantitative PCR (qPCR).

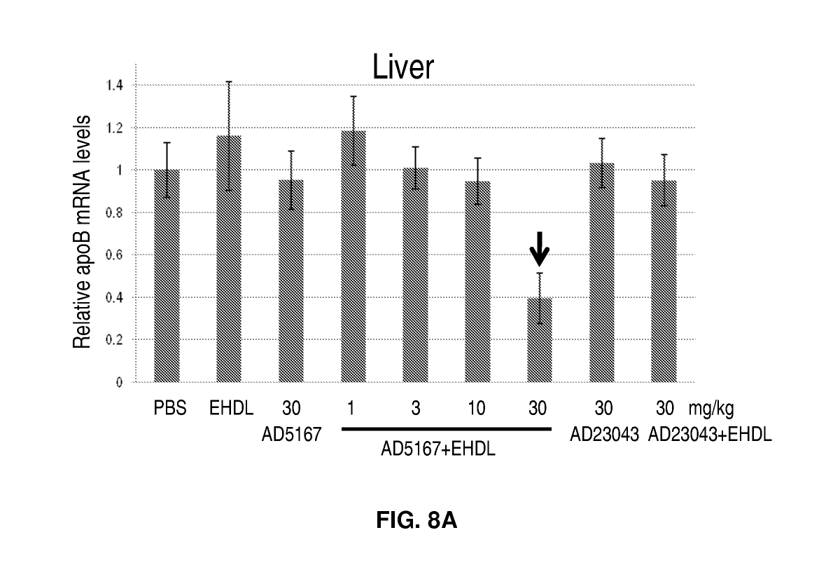

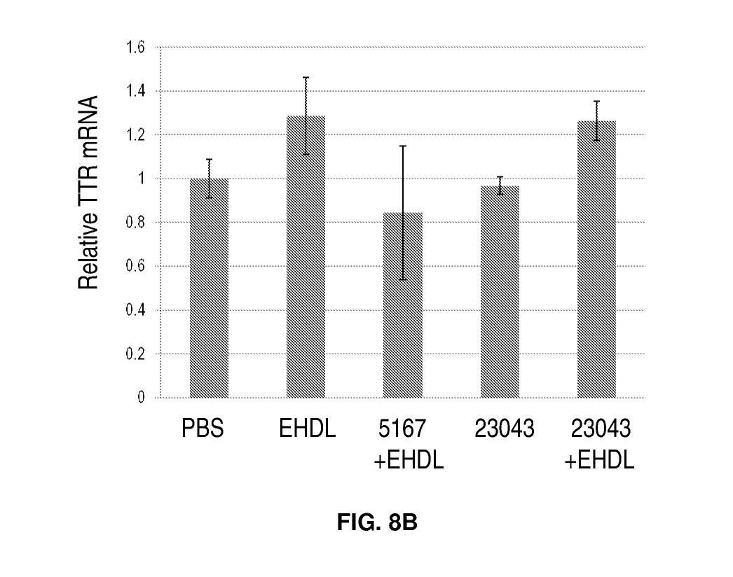

FIG. 8A shows the result of bDNA assays. As shown in FIG. 8A, AD5167 complexed with rEHDL decreased levels of ApoB mRNA by nearly 60% in liver of C57BL6 mice at a dose of 30 mg/kg. There was little or no knockdown of transthyretin (TTR) mRNA in C57BL6 mice treated with AD23043 (Cholesterol-AD18534 (TTR siRNA)) complexed with rEHDL (FIG. 8B).

As shown in FIG. 9A, ApoB100 protein level was also reduced by about 50% in C57BL6 mice at a dose of 30 mg/kg. The decrease in ApoB100 level was not observed in mice treated with AD23043 complexed with rEHDL.

Dose response was not observed.

Reduction of ApoB in plasma by AD5544 is shown in FIG. 9B.

### Example 6

### ApoE3-Based Reconstituted HDL Complexed with ApoB dsRNA Inhibited ApoB Gene Expression In Vivo

The chemically modified ApoB siRNA (AD5544) was complexed with an ApoE-HDL and the ability of the complex to inhibit ApoB expression was tested in vivo.

ApoE-based rHDL (called rEHDL) was prepared from recombinant ApoE3 from E. coli or HEK293 cells. The rEHDL was then complexed with AD5544 as described above.

C57BL6 male mice (8 weeks old, n=4) were administered 30 mg/kg AD5167/EHDL or AD5544/EHDL, by intravenous administration in a single bolus dose. Mice were then fasted overnight (−14 hours) before being sacrificed at 48 h post-injection. Plasma apoB protein levels were determined by Western blot analysis. Liver and jejunum apoB mRNA levels were determined by bDNA assay and quantitative PCR (qPCR).

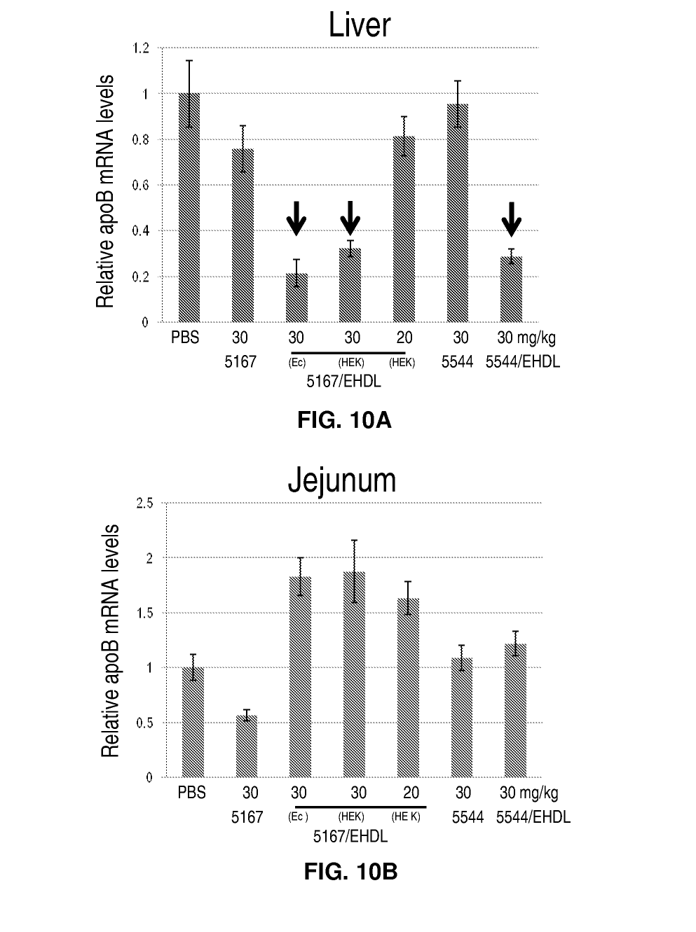

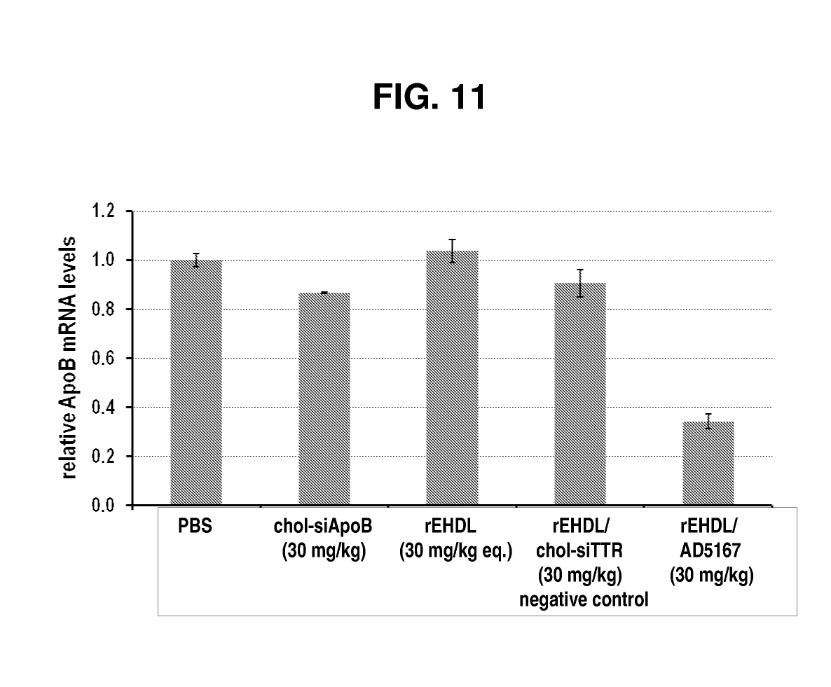

FIGS. 10A and 10B show the results of bDNA assays. There was little or no knockdown of jejunum apoB mRNA by AD5167 in C57BL6 mice. As shown in FIG. 10A, AD5167 complexed with rEHDL expressed from E. coli was as effective as the same formulation with ApoE expressed from HEK293 in inhibition of ApoB mRNA in liver of C57BL6 mice. These results indicate that rEHDL does not require glycosylation for delivery effect. As shown in FIG. 11, the knock-down effect of rEHDL/AD5167 was specific for ApoB.

Also as shown in FIG. 10A, the chemically modified AD5544 complexed with rEHDL consistently decreased levels of ApoB mRNA by about 60-80% in liver of C57BL6 mice at a dose of 30 mg/kg. There was little or no knockdown of jejunum apoB mRNA by AD5544 in C57BL6 (FIG. 10B).

### Example 7

### ApoE3-Based Reconstituted HDL Complexed with apoB dsRNA Resulted in Decreased apoB Protein Levels and Decreased Cholesterol Levels in Plasma of Mice

The chemically modified ApoB siRNA (AD1567) was complexed with rEHDL, and the effect of the complex on plasma ApoB protein levels and cholesterol levels was assayed in vivo.

C57BL6 male mice were administered 30 mg/kg AD5167 or 30 mg/kg rEHDL/AD5167, by intravenous administration in a single bolus dose. Mice were then fasted overnight (˜14 hours) before being sacrificed at 48 h post-injection. Plasma apoB protein levels were determined by Western blot analysis, and serum cholesterol levels were measured using a colorimetric assay essentially as described by Roeschlau et al. (Clin. Chem. Clin. Biochem. 12:226, 1974).

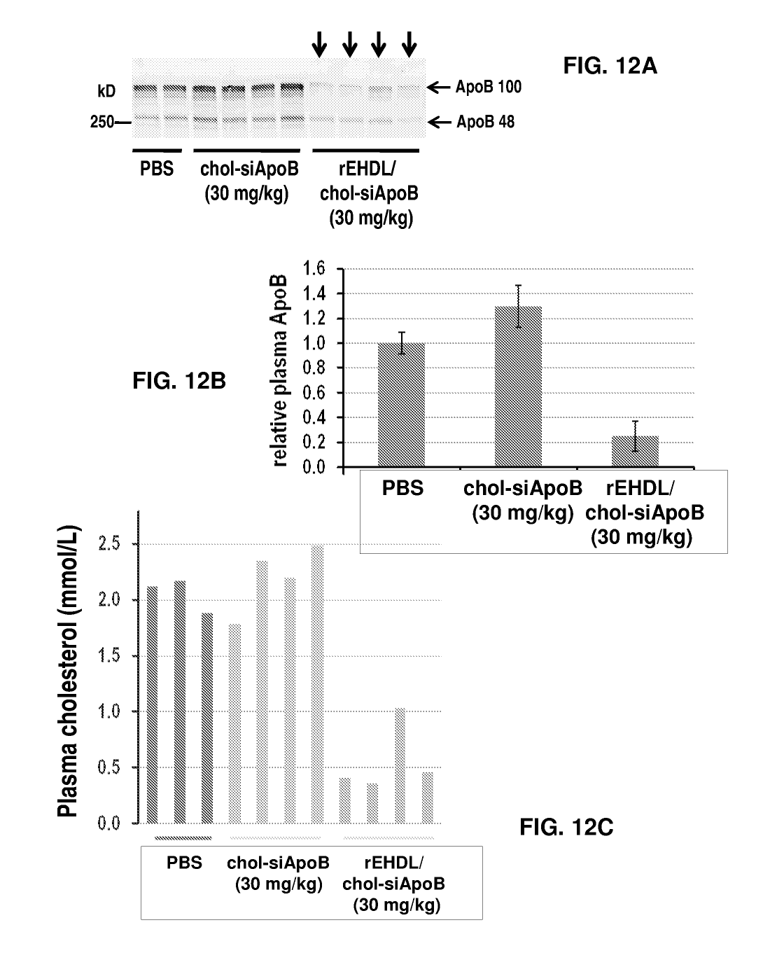

FIGS. 12A and 12B show the results of Western blot assays. There was a significant reduction in ApoB protein levels following administration of rEHDL complexed siRNAs, but not following administration of uncomplexed siRNAs. Consistent with this observation, FIG. 18C illustrates that plasma cholesterol levels were significantly reduced following administration of rEHDL/AD5167, but not following administration of uncomplexed AD5167.

### Example 8

### ApoE3-Based Reconstituted HDL Complexed with dsRNAs Targeting PCSK9 (Proprotein Convertase Subtilisin/Kexin Type 9) and Coagulation Factor Vii (FVII) RNAs Resulted in Decreased Pcsk9 and Fvii mRNAs, Respectively

DsRNAs targeting PCSK9 and FVII were complexed with rEHDL, and the effect of the complex on target protein levels was assayed in vivo.

C57BL6 mice were administered 30 mg/kg rEHDL/chol-siPCSK9 or 30 mg/kg rEHDL/chol-siFVII, by intravenous administration (tail vein injection) in a single bolus dose.

Chol-siPCSK9 (dsRNA Duplex D-20583) has the following sequence:

(SEQ ID NO: 18)

| | |
|---|---|
| Sense: | GccuGGAGuuuAuucGGAAdTsdTL10 |

(SEQ ID NO: 19)

| | |
|---|---|
| Antisense: | PuUfcCfgAfaUfaAfaCfuCfcAfgGfcdTsdT |

L10 is N-(cholesterylcarboxamidocaproyl)-4-hydroxyprolinol (Hyp-C6-Chol) and has the following structure:

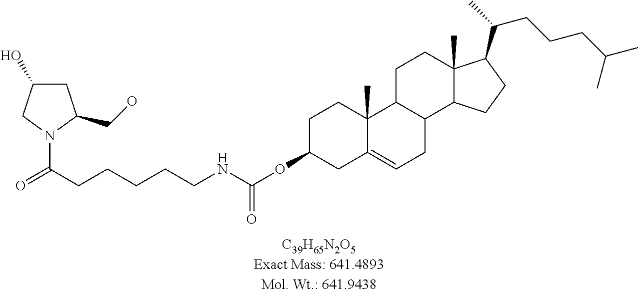

Chol-siFVII (dsRNA Duplex Name AD-18120) has the following sequence:

(SEQ ID NO: 20)

| | |
|---|---|
| Sense: | GGAUfCfAUfCfUfCfAAGUfCfUfUfACfdTsdTsL10 |

(SEQ ID NO: 21)

| | |
|---|---|
| Antisense: | GUfAAGACfUfUfGAGAUfGAUfCfCfdTsdT |

After injection, mice were fasted overnight (−14 hours), and then sacrificed at 48 h post-injection. mRNA levels from liver were determined by bDNA assay, and normalized to GAPDH mRNA levels.

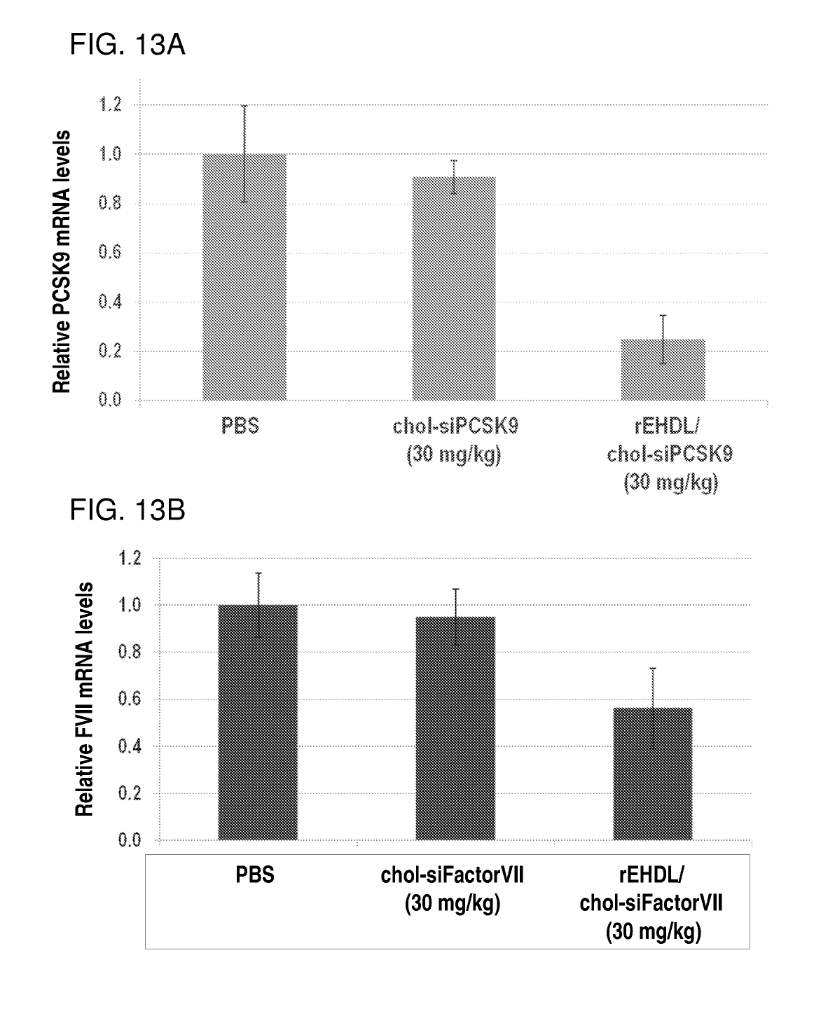

The results of the bDNA assays are shown in FIGS. 13A and 13B, which indicate that there was a significant reduction in PCSK9 and FVII mRNA levels following administration of rEHDL complexed PCSK9 and FVII siRNAs, respectively, but not following administration of uncomplexed siRNAs. rEHDL/chol-siPCSK9 decreased PCSK9 mRNA levels by about 80%, and rEHDL/chol-FVII siRNAs decreased FVII mRNA levels by about 45%. These results indicate that rEHDL complexes are generally useful for delivery of siRNAs, and are not limited for use only with siRNAs that target ApoB. The results also further support previous observations that gene expression knockdown by rEHDL complexes is specific and robust.

### Example 9

### rEHDL/chol-siRNA Complexes Appear To be Taken Up by Cells Through Endocytosis

Uptake of rEHDL/chol-siRNAs was examined in vitro in Hep3B cells. The complexes were formed as follows: ApoE-HDL was conjugated with Alexa 488 C5 maleimide from Molecular Probes at a ratio of two Alexa molecules per HDL (hereafter, “rEHDL-Alexa488”). The siRNA AD-22360.1 was conjugated with Alexa647 (hereafter, “chol-siRNA-Alexa647”).

Cells were incubated at 37° C. with each the following test samples: (i) chol-siRNA-Alexa647; (ii) rEHDL-Alexa488/chol-siRNA-Alexa647; (iii) rEHDL-Alexa488-AD5544; (iv) rEHDL/chol-siRNA-Alexa647. Each of the samples were applied to cells at a concentration of 100 nM, and live imaging of the cells by Opera™ (PerkinElmer, Waltham, Mass.) was performed for as long as 10 minutes to examine the uptake of the particles in real time.

The images indicated that uptake of uncomplexed chol-siRNA occurs by a different mechanism than uptake of rEHDL/chol-siRNA complexes. siRNAs complexed with rEHDL appeared at the plasma membrane and in vesicular/punctate structures within the cells. This result suggests that rEHDL/chol-siRNAs are taken up by the cell through receptor mediated endocytosis, possibly by binding to SR-BI, a receptor that mediates uptake of HDL.

### Example 10

### Characterization of rEHDL Particles

rEHDL particles were prepared by solubilizing phospholipid (DMPC) in sodium cholate solution (20 mg/mL), 37° C., such that the final cholate:DMPC ration was 3:1. When the DMPC mixture was solubilized, ApoE was added to final ApoE:DMPC ratio of 1:192. This mixture was incubated at 37° C. for at least one hour. The resulting mixture was dialyzed against 1×PBS using a molecular weight cutoff of less than 30K. After extensive dialysis (dilution factor >1×10⁶, the resulting particles were concentrated and characterized.

To characterize the particles, the absorbance at 280 nm under denaturing conditions was measured; phosphatidyl choline analysis was performed using a commercial assay from Wako Pure Chemical (Osaka, Japan); size exclusion analysis was performed using a Superdex 100 HR 10/30 column in 1×PBS; and size analysis was performed by dynamic light scatter (Malvern Instruments Zetasizer Nano ZS (Worcestershire, United Kingdom).

rEDHL particles were complexed with chol-siRNAs, by mixing chol-siRNA (20 mg/mL stock) with rEDHL at a final ratio chol-siRNA:rEHDL of 1.2:1. This mixture was incubated 30 minutes at 37° C. The complex was cooled to room temperature, then centrifuged 1000×G for 2 minutes. The rEHDL/chol-siRNA complexes were purified by size exclusion chromatography over a Superdex200 26x40×2 column in 1×PBS. Samples were pooled and concentrated in an Amicon Ultra-15, 30K MWCO, and then chol-siRNA concentration was measured by HPLC ion exchange. ApoE concentration was measure using a modified bicinchoninic acid (BCA) assay (Pierce, Rockford, Ill.). The BCA assay used rEHDL of known concentration as the standard (not BSA).

The molecular weight of the chol-siRNA is about 15 kD, the molecular weight of ApoE is 34.3 kD, the molecular weight of DMPC is 677.9 g/mol, and the molar ratio of chol-siRNA:ApoE:DMPC was calculated to be about 1:2:384. An efficacious dose of the rEHDL siRNA complex is about 30 mg/kg chol-siRNA, 137 mg/kg ApoE, and 521 mg/kg DMPC.

The plasma pharmacokinetics and biodistribution of rEHDL/chol-siRNA complexes has also been determined in mice using ³²P-labeled chol-siRNA. The plasma half-life of rEHDL complexes was determined to be about 10 minutes, indicating that the complexes are rapidly cleared. Most of the rEHDL/chol-siRNA is taken up by the liver, and minor fractions are absorbed by the gut and kidneys.

### Example 11

### Efficacy Study of rEHDL/chol-siRNA Particles in Double (apoB x CETP) Transgenic Mice

To test whether rEHDL/chol-siRNA particles can deliver siRNA to the liver in the animal model that has lipoprotein compositions similar to those of humans and to evaluate the translational potential of such particles in the clinic, an efficacy study of rEHDL/chol-AD5544 particles was performed in double (apoB x CETP) transgenic mice.

In order to better mimic the plasma lipoprotein concentrations found in human (i.e., VLDL, LDL and HDL), double transgenic mice expressing human ApoB and human CETP were created. As shown in Table 6, the percentage of total cholesterol in VLDL, LDL and HDL in mice expressing both human apoB and CETP was similar to that of human (11%, 64% and 25%, respectively, in male mice; 6%, 60% and 34%, respectively, in human). The percentage of total cholesterol within the LDL was much higher in the double transgenics than in the non-transgenic mice, mice expressing human CETP alone, or mice expressing human apoB alone. In addition, HDL cholesterol levels were reduced in the double transgenic animals or animals expressing human apoB or CETP alone.

TABLE 6
Lipid levels in double (apoBxCETP) transgenic mice

| | | | | | |
|---|---|---|---|---|---|
| | | Total | VLDL | LDL | HDL |
| Species | Triglycerides | Cholesterol | Cholesterol | Cholesterol | Cholesterol |

Plasma lipoprotein cholesterol concentrations (mg/dL)

| | | | | | |
|---|---|---|---|---|---|
| Human¹ | 110 ± 13.1 | 192 ± 11.5 | 12.5 ± 2.7 | 101 ± 14.1 | 59 ± 15 |
| Monkey¹ | 90 ± 7.2 | 125 ± 11.1 | 7.0 ± 1.6 | 57 ± 1.5 | 70 ± 5.9 |
| Mouse¹ | 20 ± 3.9 | 92 ± 9.6 | 4.5 ± 1.3 | 15 ± 6.8 | 79 ± 11 |
| TG (apoBxCETP) M² | 240 ± 16 | 113 ± 7 | | | 23 ± 2 |
| TG (apoBxCETP) F² | 146 ± 16 | 127 ± 4 | | | 30 ± 2 |

% of total cholesterol²

| | | | |
|---|---|---|---|
| Non-Tg M (normal mice) | 2 | 13 | 84 |
| CETP−/− M | 11 | 24 | 65 |
| ApoB−/− M | 2 | 31 | 68 |
| TG (apoBxCETP) M | 11 | 64 | 25 |
| Human | 6 | 60 | 34 |

¹Greve J. et al., J Lip Res. 34, 1367 (1993)
²Grass D. S. et al., J. Lip Res. 36, 1082 (1995)

To examine whether effective silencing of liver apoB can be achieved by rEHDL/chol-siRNA particles, double transgenic mice (n=4) were administered 10 or 33 mg/kg AD5544 complexed with rEHDL, or 33 mg/kg AD5544 without rEHDL formulation, by intravenous administration in a single IV bolus dose. Mice were sacrificed at 48 h post-injection. AD5544 is a chemically modified apoB siRNA duplex and has human/mouse cross-reactivity. rEHDL/AD5544 has the following sequences:

| (SEQ ID NO: 16) | |
|---|---|
| Sense: | GGAAUCuuAuAuuuGAUCcAAs-L10 |
| (SEQ ID NO: 17) | |
| Antisense: | uuGGAUcAAAuAuAAGAuUCcscsU |

The struction of rEHDL (also called “L10”) is described above.

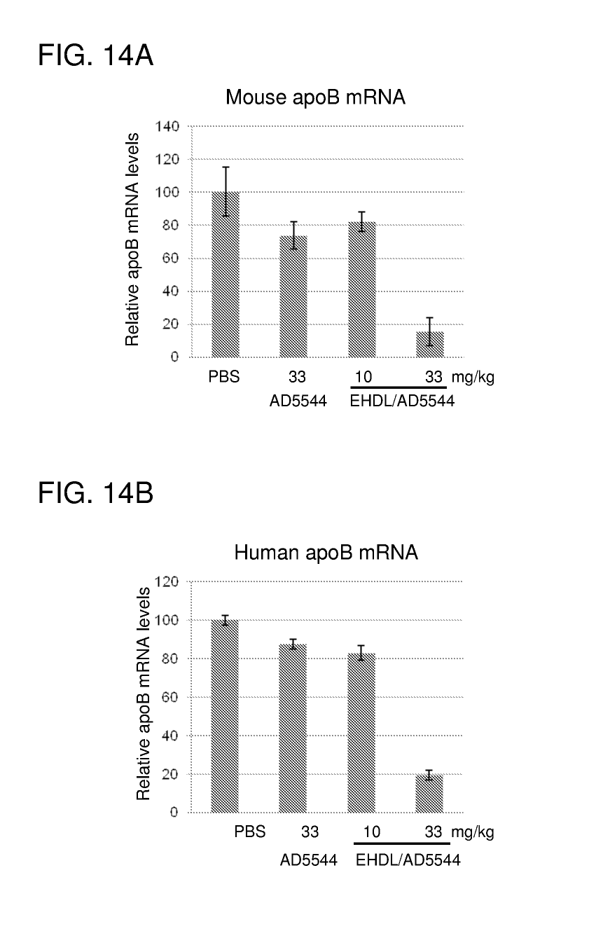

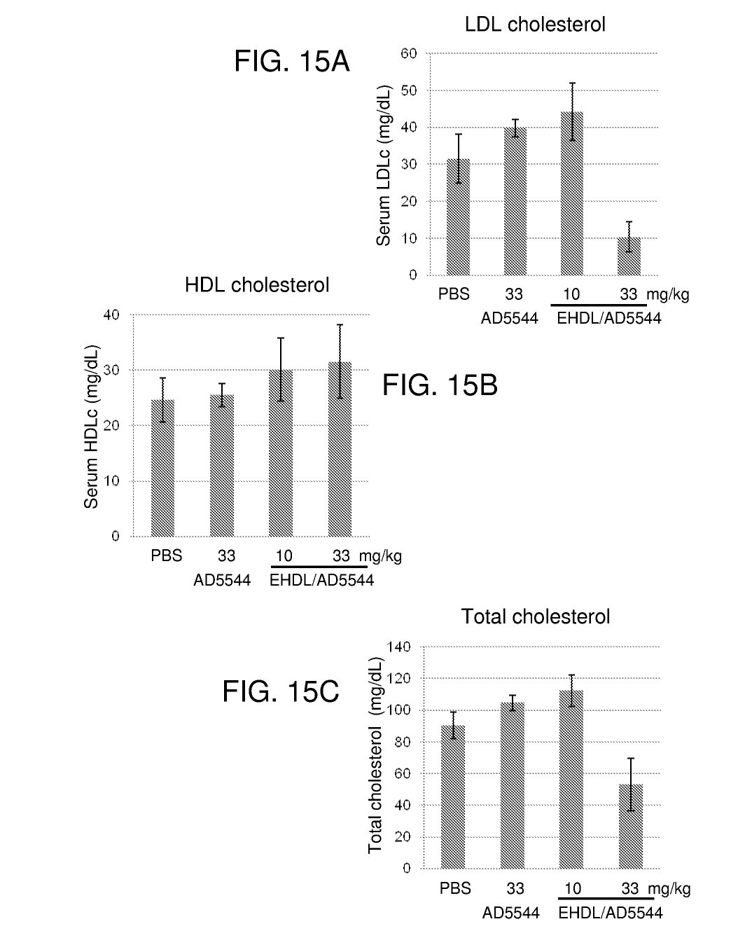

As shown in FIGS. 14A and 14B, rEHDL/AD5544 decreased the levels of both mouse and human apoB mRNA in the liver by about 80-85% at a dosage of 33 mg/kg. This knockdown level is nearly identical to that in C57BL6 mice. This result demonstrates effective silencing in transgenic mice with lipoprotein profile similar to humans, such as human-like high levels of LDL and low levels of HDL. This result also suggests that high levels of endogenous LDL do not compete with rEHDL for liver uptake. No enhancement of apoB mRNA knockdown was observed in mouse jejunum.

As shown in FIGS. 15A and 15C, serum LDLc and total cholesterol levels were lowered to about 33% and 60%, respectively, in double transgenic mice administered 33 mg/kg rEHDL/AD5544, as compared to the serum LDLc and total cholesterol levels in controlled mice administered PBS. FIG. 15B shows that HDL cholesterol levels were not affected in double transgenic mice administered rEHDL/AD5544.

Histology showed severe liver necrosis and inflammatory response in spleen at 50 mg/kd dose of the rEHDL/AD5544 particle. Minimal liver and mild splenic histopath effects were observed with a dose of 30 mg/kg.

### Example 12

### Pharmacokinetics (PK) Study of rEHDL/AD5544

C57BL6 mice were administered 30 mg/kg rEHDL/AD5544, by intravenous administration in a single bolus dose. Plasma, liver and spleen concentrations of lipoprotein formulated AD5544 in C57BL6 mice were measured at different time points post-injection (up to 24 hours). Plasma, liver and spleen concentrations of AD5544 showed a good correlation. Plasma concentration of AD5544 was about 10-fold higher than the concentration of AD5544 in liver and about 15-20 fold higher than the concentration of AD5544 in spleen at various time points. Only approximately 10% of AD5544 accumulated in the liver for the first 4 hours post dose. Distribution of AD5544 in plasma, liver and spleen appeared to be at steady state for the first four hours post-injection. Fast clearance of AD5544 after the first four hours post-injection was observed.

To examine whether rEHDL formulation althers the plasma PK parameters of AD5544, mice were administered 30 mg/kg AD5544 complexed with rEHDL or 100 mg/kg AD5544 without rEHDL formulation. Plasma PK parameters in mice administered 100 mg/kg AD5544 without rEHDL formulation were normalized to the dosage of 30 mg/kg for the purpose of comparison. The result showed that plasma PK parameters (Tₘₐₓ (h), AUC₀₋ₜ (h·ng/mL), AUC_{inf} (h·ng/mL), t_{1/2} (h), CL (mL/h/kg), Vss (mL/kg), and MRT₀₋ₜ (h)) of AD5544 complexed with rEHDL was very similar to those of AD5544 alone. However, the efficacy of AD5544 was much improved when complexed with rEHDL, suggesting that factors other than plasma exposure drive better delivery of rEHDL-complexed siRNAs to the liver.

Other embodiments are in the claims.

1. A composition comprising a particle, wherein the particle comprises
(a) at least one oligonucleotide and
(b) at least one recombinant Apolipoprotein E (ApoE), and wherein the particle is substantially devoid of other apolipoproteins.

2. The composition of claim 1 wherein said oligonucleotide is conjugated to a lipophile.

3. The composition of claim 2 wherein the lipophile-conjugated oligonucleotide comprises a double stranded oligonucleotide.

4. The composition of claim 1 wherein the ApoE is an ApoE3 isoform.

5. (canceled)

6. The composition of claim 5 claim 1 wherein the ApoE is reconstituted with at least one amphiphilic agent.

7. The composition of claim 6 wherein the amphiphilic agent is a phospholipid.

8. The composition of claim 7 wherein the phospholipid is selected from the group consisting of dimyristoyl phosphatidyl choline (DMPC), dioleoylphosphatidylethanolamine (DOPE), palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), -phosphatidylethanolamine (POPE), dioleoyl-phosphatidylethanolamine 4-(N-maleimidomethyl)-cyclohexane-1-carboxylate (DOPE-mal), and combinations thereof.

9. The composition of claim 1 wherein the ApoE is reconstituted high density lipoprotein (rEHDL).

10. (canceled)

11. The composition of claim 1, further comprising one or more of a Low Density Lipoprotein (LDL), a Very Low Density Lipoprotein (VLDL), an Intermediate Density Lipoprotein (IDL), and a chylomicron.

12. (canceled)

13. (canceled)

14. The composition of claim 1, wherein the particle comprises at least about 1 to 3 oligonucleotides, at least about 3 to 5 oligonucleotides, at least about 5 to 8 oligonucleotides, at least about 8 to 10 oligonucleotides, at least about 10 to 15 oligonucleotides, or at least about 15 to 20 oligonucleotides.

15-18. (canceled)

19. The composition of claim 3 wherein said double stranded oligonucleotide comprises a sense strand and an antisense strand, wherein each of said strands comprises 18 to 30 nucleotides and said strands form a complementary double stranded region of 18 to 30 basepairs.

20. The composition of claim 19, wherein said complementary double stranded region has 0, 1, 2, or 3 nucleotide single stranded overhangs on at least one terminal end.

21. The composition of claim 1, wherein said oligonucleotide comprises at least one non-phosphodiester linkage.

22. The composition of claim 1, wherein said oligonucleotide comprises at least one modified nucleoside.

23. The composition of claim 2 wherein the lipophile conjugate is a cholesterol moiety.

24. (canceled)

25. A method for selectively targeting and/or delivering an oligonucleotide to a mammalian tissue comprising contacting a mammal with said oligonucleotide, wherein said oligonucleotide has been preassembled with an ApoE.

26. The method of claim 25 wherein said mammalian tissue is liver.

27. The method of claim 25 wherein the oligonucleotide is a dsRNA.

28. (canceled)

29. The method of claim 27 wherein said dsRNA targets ApoB.

30. A method of reducing expression of a gene in mammalian tissue in vivo comprising contacting said tissue with the composition of claim 1.
